(19) United States
(12) Patent Application Publication
(10) Pub. No.: US 2003/0065015 A1
(43) Pub. Date: Apr. 3, 2003

(54) Novel quarternary ammonium compositions coupled with facilitating anions and their use in kits, as well as their use in preventing and treating certain conditions

(76) Inventors: Marvin B. Bacaner, Minneapolis, MN (US); Maurice M. Kreevoy, Minneapolis, MN (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/144,909

(22) Filed: May 13, 2002

Related U.S. Application Data
(60) Provisional application No. 60/290,456, filed on May 11, 2001.

(51) Int. Cl. A61K 31/44

(52) U.S. Cl. 514/357

(57) ABSTRACT

This invention is directed to novel pharmaceutical compositions and kits comprising quaternary ammonium salts as the active ingredient and facilitating anions for their absorption into the bloodstream and transportation to the end sites, as well as methods which use such compositions and kits for preventing and/or treating conditions for which the drugs are used to treat.

### CROSS REFERENCE TO RELATED APPLICATIONS

This application claims priority from U.S. Provisional Application Serial No. 60/290,456, filed May 11, 2001, the entire text of which is hereby incorporated herein by reference.

### FIELD OF THE INVENTION

This invention relates to novel compositions and kits comprising a pharmaceutically active quaternary ammonium cation, for example, 2-PAM, pyridostigmine or neostigmine, and a facilitating anion with or without other active agents such as, for example, atropine. This invention also relates to the use of such compositions and kits to prevent and/or treat various conditions, including exposure to organophosphorus cholinesterase inhibitors and myasthenia gravis.

### BACKGROUND OF THE INVENTION

A variety of quaternary ammonium salts are pharmaceutically active.

The quaternary ammonium salt 2-[(hydroxyimino)methyl]-1-methylpyrdinium chloride (Pralidoxime chloride; 2-PAM chloride) is a cholinesterase reactivator which counteracts the toxic effect of organophosphorus cholinesterase inhibitors such as sarin nerve gas and other organophosphorus compounds that are used, e.g., as agricultural pesticides. For example, organophosphorus cholinesterase inhibitors such as sarin nerve gas are rapidly lethal upon inhalation, ingestion or transdermal absorption. By reactivation of cholinesterase, 2-PAM chloride is an effective antidote against these toxic agents. However, unless an antidote is administered immediately, death from exposure to sarin nerve gas ordinarily occurs within minutes.

2-PAM chloride is conventionally administered parenterally rather than by ingestion since it is not effectively absorbed into the bloodstream from the gastrointestinal tract or across the other lipid membranes. Further, exposure to neurotoxic organophosphorus compounds most often occurs in the field, e.g., in combat where an organophophorus gas is disseminated as an anti-personnel weapon, or in agricultural applications where an organophosphorus pesticide is inadvertently sprayed or drifts into an area where agricultural laborers are present. Thus, field exposure makes intravenous administration difficult, unreliable, sometimes impossible, and at best inefficient. Intravenous administration can be exceptionally difficult under combat conditions, but may be equally difficult in an agricultural setting where exposure is unexpected and rescue may be disorganized.

The administration of 2-PAM to nerve gas victims would be greatly facilitated by the availability of a 2-PAM composition suitable for oral administration with prompt bioavailability. However, available formulations based on the chloride salt have proven ineffective when administered by ingestion, due to the very limited absorbability of 2-PAM chloride through the gastrointestinal wall. Thus, there is an unfulfilled need in the art for an alternative antidote to neurotoxic organophosphorus compounds, e.g. an improved 2-PAM formulation, suitable for and effective by oral ingestion.

Pyridostigmine, systematic name, 3-{[(dimethylamino)carbonyl]oxy}-1-methylpyridinium ion is used as a potentiating agent for 2-PAM if taken before exposure to nerve gas. Use of pyridostigmine allows for lower 2-PAM dosages and more effective use of 2-PAM as discussed herein. Pyridostigmine is available in a pyridostigmine bromide formulation. For example, pyridostigmine bromide was given to Desert Storm troops in small quantities for oral ingestion. However, bromide ion has unwanted side effects and is thought to be a potential source of the Gulf War Syndrome for the soldiers who took pyridostigmine bromide as a potentiating agent for 2-PAM. Further, it is believed that pyridostigmine is not well absorbed into the body when orally taken as pyridostigmine chloride, thus requiring higher doses to be applied. Therefore, a further need exists for a more effective pyridostigmine composition suitable for oral administration with prompt bioavailability.

Neostigmine, 3-[[(Dimethylamino)carbonyl]oxy]-N,N,N-trimethylbenzenaminium, is a relatively fast-acting and potent drug used in the treatment of myasthenia gravis, an autoimmune condition which involves muscle weakness caused by faulty transmission of nerve impulses across the neuromuscular junction. By prolonging the nerve impulses, neostigmine improves muscle strength, even though it does not cure the disease. In severe cases neostigmine may be prescribed in conjunction with corticosteroids or other drugs. Neostigmine is also used to relieve urinary retention or temporary paralysis of the bowel (paralytic ileus and/or urinary retention or pseudo-obstruction of the bowel) that is often seen postoperatively.

Neostigmine is currently available as neostigmine bromide; however, the side effects from the bromide are undesirable. Moreover, available formulations based on the bromide salt have proven ineffective when administered by ingestion, due to the very limited absorbability of the compound through the gastrointestinal wall and through the neuromuscular junction. Thus, there is an unfulfilled need in the art for an ingestible formulation of neostigmine, which can be used as an alternative treatment to treat myasthenia gravis and/or pseudo bowel obstruction. Such conditions currently require an injection of neostigmine, which could be obviated in favor of oral administration with the proper formulation that would allow gastrointestinal uptake of suitable quantities of neostigmine.

The interstitial connective tissue in the peripheral nerve that separates the individual nerve fibers of a vertebrate is referred to as the endoneurium, and can be visualized as an insulative medium in which conductive wires are embedded. Blood vessels in the endoneurium of peripheral nerves are comparable to those of the central nervous system and are lined by a continuous endothelium, made up of capillary endothelial cells, with intercellular tight junctions of high electrical resistance (100 ohm/cm). Together with the perineurium, a connective tissue sheath immediately surrounding the fascicles of nerve fibers, the vessels form a blood-nerve barrier to regulate the microenvironment of the endoneurium of the nerve.

The blood-nerve barrier is an effective barrier to both endogenous and exogenously-administered blood components, including peptides, proteins and other large macromolecules, as well as to ions and water-soluble non-electrolytes. This protects the endoneurial microenvironment from rapid changes in the composition of the blood or of the extraneural spaces. Also, alterations in the blood nerve barrier integrity are implicated in a number of peripheral nerve disorders, such as those caused by diabetes mellitus, toxins, infection and autoimmune disorders.

However, the ability of the blood-nerve barrier to protect the nervous system from exogenous substances has impeded the development of therapies for a wide variety of neural pathologies and disorders. Thus, a continuing need exists for methods to increase the permeability of the blood nerver barrier to bioactive substances. In applications where speed of treatment is critical (e.g., administration of 2-PAM for cholinesterase inhibition), the ability to quickly cross the blood nerve barrier is of great benefit. Further, the ability to transport more 2-PAM, pyridostigmine or neostigmine ions across the blood nerve barrier reduces the dosage of the ion of interest needed to provide a therapeutic effect in the subject. This not only reduces the cost of treatment, but may also limit toxic side effects that are typically associated with certain quaternary ammonium salts.

### SUMMARY OF THE INVENTION

This invention, in the broadest sense, provides for improved methods of transferring quaternary ammonium cations such as 2-PAM (Pralidoxime, 2-[(hydroxyimino)methyl]-1-methylpridinium) ions, pyridostigmine ions or neostigmine ions across biological membranes in animals, particularly humans, by use of facilitating anions.

Briefly, therefore, the present invention is directed to a pharmaceutical combination useful for treating exposure to a cholinesterase inhibitor. The combination comprises a 2-PAM cation or a source of a 2-PAM cation and a facilitating anion or a source of facilitating cation. The combination is further characterized in that the facilitating anion is less hydrophilic than a chloride anion and the 2-PAM cation or the source of the 2-PAM cation and the facilitating anion or the source of the facilitating anion together are present in the pharmaceutical combination in a therapeutically effective amount.

The present invention is further directed to a pharmaceutical combination useful for treating exposure to a cholinesterase inhibitor. The combination comprises a 2-PAM cation or a source of a 2-PAM cation and an anion or a source of an anion, wherein the anion is selected from the group consisting of (C₁₀-C₃₀)alkylsulfate anions, (C₁₀-C₃₀) alkylsulfonate anions, (C₆-C₁₂)alkylsulfosuccinate anions, salicylate anions, (C₁-C₃₀)alkylsalicylate anions, (C₁₀-C₃₀)alkylphosphate anions, di(C₁-C₁₋₂)alkylphosphate anions, di(C₁₀-C₃₀)alkanoylphosphatidate anions, (C₈-C₂₂)alkylmaleate anions, di(C₄-C₁₂)alkylmaleate anions, α-keto (C₉-C₂₁)carboxylate anions, α-hydroxy (C₉-C₂₁)carboxylate anions, (C₁₂-C₂₂)alkylmalonate anions, and (C₁-C₁₈)alkylpseudo-icosahedral carborane anions.

The present invention is further directed to a pharmaceutical combination useful for treating exposure to a cholinesterase inhibitor. The combination comprises a 2-PAM cation or a source of a 2-PAM cation, a facilitating anion or a source of a facilitating anion, and an anticholinergic agent or a source of an anticholinergic agent. The combination is further characterized in that the facilitating anion is less hydrophilic than a chloride anion and that the 2-PAM cation or the source of the 2-PAM cation and the facilitating anion are, in combination, suitable for oral ingestion. Further, the 2-PAM cation or the source of the 2-PAM cation and the facilitating anion or the source of the facilitating anion are capable of forming a mixture comprising a 2-PAM cation and a facilitating anion within the gastrointestinal tract of a subject upon ingestion of the combination by the subject. Still further, the 2-PAM cation or the source of the 2-PAM cation and the facilitating anion or the source of the facilitating anion together are present in the pharmaceutical combination in a therapeutically effective amount.

The present invention is further directed to a pharmaceutical combination useful for potentiating clearance of a cholinesterase inhibitor. The combination comprises a pyridostigmine cation or a source of a pyridostigmine cation and a facilitating anion or a source of facilitating cation. The combination is further characterized in that the facilitating anion is less hydrophilic than a chloride anion and the pyridostigmine cation or the source of the pyridostigmine cation and the facilitating anion or the source of the facilitating anion together are present in the pharmaceutical combination in a therapeutically effective amount.

The present invention is further directed to a pharmaceutical combination useful for potentiating clearance of a cholinesterase inhibitor. The combination comprises a pyridostigmine cation or a source of a pyridostigmine cation and an anion or a source of an anion selected from the group consisting of (C₁₀-C₃₀)alkylsulfate anions, (C₁₀-C₃₀)alkylsulfonate anions, (C₆-C₁₂)alkylsulfosuccinate anions, salicylate anions, (C₁-C₃₀)alkylsalicylate anions, (C₁₀-C₃₀)alkylphosphate anions, di(C₁-C₁₂)alkylphosphate anions, di(C₁₀-C₃₀)alkanoylphosphatidate anions, (C₈-C₂₂)alkylmaleate anions, di(C₄-C₁₂)alkylmaleate anions, α-keto (C₉-C₂₁)carboxylate anions, α-hydroxy (C₉-C₂₁)carboxylate anions, (C₁₂-C₂₂)alkylmalonate anions, and (C₁-C₁₈)alkylpseudo-icosahedral carborane anions.

The present invention is further directed to a pharmaceutical combination useful for potentiating clearance of a cholinesterase inhibitor. The combination comprises a potentiating agent or a source of a potentiating agent and a facilitating anion or a source of a facilitating anion. The combination is further characterized in that the facilitating anion is less hydrophilic than a chloride anion and that the potentiating agent or the source of the potentiating agent and the facilitating anion or the source of the facilitating agent together are present in the pharmaceutical combination in a therapeutically effective amount.

The present invention is further directed to a pharmaceutical combination useful for treating nerve conditions such as pseudoobstruction of the bowel, paralytic ileus and/or urinary retention, or myasthenia gravis. The combination comprises a neostigmine cation or a source of a neostigmine cation and a facilitating anion or a source of facilitating cation. The combination is further characterized in that the facilitating anion is less hydrophilic than a bromide anion and the neoostigmine cation or the source of the neostigmine cation and the facilitating anion or the source of the facilitating anion together are present in the pharmaceutical combination in a therapeutically effective amount.

The present invention is further directed to a pharmaceutical combination useful for treating nerve conditions such as pseudoobstruction of the bowel, paralytic ileus and/or urinary retention, or myasthenia gravis. The combination comprises a neostigmine cation or a source of a neostigmine cation and an anion or a source of an anion selected from the group consisting of (C₁₀-C₃₀) alkylsulfate anions, (C₁₀-C₃₀)alkylsulfonate anions, (C₆-C₁₂)alkylsulfosuccinate anions, salicylate anions, (C₁-C₃₀)alkylsalicylate anions, (C₁₀-C₃₀) alkylphosphate anions, di (C₈-C₁₂)alkylphosphate anions, di (C₁₀-C₃₀) alkanoylphosphatidate anions, (C₈-C₂₂)alkylmaleate anions, di(C₄-C₁₂)alkylmaleate anions, α-keto (C₉-C₂₀)carboxylate anions, α-hydroxy (C₉-C₂₁) carboxylate anions, (C₁₂-C₂₂) alkylmalonate anions, and (C₁-C₁₈)alkylpseudo-icosahedral carborane anions.

Still further the present invention is directed to novel pharmaceutical compositions comprising 2-PAM di(2-ethylhexyl)sulfosuccinate, 2-PAM salicylate, 2-PAM di(2-ethylhexyl)phosphate, 2-PAM lauryl sulfate, 2-PAM hexadecylsulfonate, 2-PAM acetylsalicylate, pyridostigmine hexadecylsulfonate, pyridostigmine di(2-ethylhexyl)sulfosuccinate, pyridostigmine salicylate, pyridostigmine di(2-ethylhexyl)phosphate, pyridostigmine lauryl sulfate, pyridostigmine acetylsalicylate, neostigmine hexadecylsulfonate, neostigmine di(2-ethylhexyl)sulfosuccinate, neostigmine salicylate, neostigmine di(2-ethylhexyl)phosphate, neostigmine lauryl sulfate, and neostigmine acetylsalicylate.

Still further, the present invention is directed to novel methods for treating exposure to a cholinesterase inhibitor, potentiating clearance of a cholinesterase inhibitor and treating nerve conditions such as pseudoobstruction of the bowel, paralytic ileus and/or urinary retention, or myasthenia gravis using the above pharmaceutical combinations.

Other objects and features of this invention will be in part apparent and in part pointed out hereinafter.

### DETAILED DESCRIPTION OF THE PREFERRED EMBODIMENTS

Past efforts to orally administer quaternary ammonium drugs (such as 2-PAM, pyridostigmine and neostigmine) have failed due to poor and/or unpredictable absorption in the gastrointestinal tract. For example, when conventional quaternary ammonium-containing drugs have been administered orally, the active component of the drugs (i.e., the quaternary ammonium cation) has typically exhibited poor and/or unpredictable uptake in the gastrointestinal tract. This has made oral self-administration difficult, thereby largely limiting the use of the drugs to parenteral administration.

Unlike previous oral formulations of quaternary ammonium compounds, the pharmaceutical compositions and kits of the present invention are uniquely adapted for oral administration. In addition, they often tend to exhibit superior activity, time for onset of action, potency, safety, and/or therapeutic effectiveness relative to conventionally used quaternary ammonium formulations. In many instances, the compositions and kits of this invention are especially advantageous because they may be self-administrated as needed, for example, at a person's residence or place of work, without the assistance of a health care professional.

Hypothesized Mechanisms of Quaternary Ammonium Action

In accordance with the present invention, it has been discovered that therapeutic quaternary ammonium cations can be transferred across a lipid barrier when in the company of one or several facilitating anions. In particular, the use of a quaternary ammonium cation in combination with a facilitating anion that is less hydrophilic than a chloride, bromide or tosylate anion has been found to result in improved and increased transfer of the quaternary ammonium cation across the biological membranes of interest.

In the aqueous contents of the gastrointestinal tract (particularly the stomach and intestine), orally administered quaternary ammonium compounds are ionized to the quaternary ammonium cation and a corresponding anion. To provide the desired therapeutic effect, however, the quaternary ammonium cation must be absorbed from the aqueous contents of the gastrointestinal tract through the lipid phase mucosa of the gastrointestinal tract into the blood, and then transferred from the blood to the target cells. Absorption of the quaternary ammonium cation from the gastrointestinal tract into the blood requires that the hydrophilic quaternary ammonium cation cross the lipophilic lipid phase boundary of the gastrointestinal tract. However, macroscopic quantities of ions can only be transferred from one phase to another in neutral combinations because the uncompensated transfer of an electrically charged ion is energetically very unfavorable; and, otherwise, the phases become electrically charged.

In accordance with the present invention, it has been discovered that absorption (or crossing of the lipid phase boundary) of the quaternary ammonium cation from the gastrointestinal tract can be improved if the quaternary ammonium cation is combined with one or more suitable types of anions (i.e., facilitating anions) resulting in a quaternary ammonium-cation/facilitating-anion combination that is more lipophilic, or less hydrophilic, than the quaternary ammonium bromide, chloride or tosylate salt. Hydrophilicity of an anion may be reduced, for example, by spreading the negative charge over a number of atoms, or by attaching an uncharged, non-polar or weakly polar residue, such as an alkyl group to the anion.

It is hypothesized that the quaternary ammonium cation and the facilitating anion in the gastrointestinal tract can exist in the form of separate ions, ion pairs, micelles, or otherwise. When the quaternary ammonium cation enters the lipid phase, however, it is believed to exist as a quaternary ammonium-cation/facilitating-anion combination in the form of ion pairs and/or higher ion aggregates, such as inverse micelles. These quaternary ammonium-cation/facilitating-anion combinations possess a neutral or substantially neutral charge. In addition, these quaternary ammonium-cation/facilitating-anion combinations are more lipophilic, or less hydrophilic, than the chloride salt of the quaternary ammonium cation, and preferably more lipophilic, or less hydrophilic, than the bromide salt of the quaternary ammonium cation. Generally, the octanol/water partition coefficient for an anion coupled with the quaternary ammonium cation may be used as a predictor of whether the anion may function as a facilitating anion in the present invention. For example, it has been found that suitable facilitating anions of the present invention typically have a higher octanol/water partition coefficient than the quaternary ammonium cation coupled with the chloride anion (for 2-PAM) and the bromide ion (for pyridostigmine and neostigmine).

It is believed that the facilitating anions used in the present invention should have a widespread charge distribution over the molecule, such that when it aggregates with the quaternary ammonium cation, the combination is as non-polar as possible. One such anion would be 2,4,6 trinitrophenol; however, this anion is known to be toxic. Non-toxic anions capable of forming about non-polar or nearly non-polar aggregates are the focus of this invention.

When the quaternary ammonium cation and facilitating anion are ingested and ionized in the absence of a neutralizing agent, the HCl present in the stomach converts a portion of the ionized anions to the corresponding conjugate acid of the anions, which is then largely absorbed by the lipid mucosa in the intestine. As these anions are converted to their conjugate acid form and absorbed, additional anions are then converted to their conjugate acid, and, in turn, absorbed in the intestine. If the anions are too readily converted to their conjugate acid form and/or the pH of the gastrointestinal tract is too low, the hydrophilic chloride anions will effectively be the only anions available for combination with the quaternary ammonium cation (if tosylate anions are also present, they too will be converted into their conjugate acid, p-toluenesulfonic acid, and, in turn, absorbed in the intestine). Because the quaternary ammonium cation cannot be spacially separated from a counterion, and the chloride anions are not readily removed from the aqueous phase, the quaternary ammonium cation remains in the aqueous fluid of the stomach and intestine and is ultimately not absorbed. To reduce or eliminate this problem, a neutralizing agent may be administered to increase the pH of the stomach. It is believed that such a pH increase enhances the absorption of the quaternary ammonium cation by reducing the removal of the facilitating anion as its conjugate acid such that a larger portion of the facilitating anion remains available to form the quaternary ammonium-cation/facilitating-anion combination.

It is further hypothesized that the compositions and kits of this invention not only enhance the absorption of the quaternary ammonium cation from the gastrointestinal tract into the blood, but also enhance the permeation of the quaternary ammonium cation from the blood through the capillary walls and the target tissue (i.e., sympathetic nerve endings and ganglia). For example, the di(2-ethylhexyl)sulfosuccinate anion promotes the formation of water-in-oil emulsions. Such emulsions generally consist of droplets having an aqueous core surrounded by di(2-ethylhexyl)sulfosuccinate anions, with the anionic sulfonate groups directed inwardly toward the core center and the hydrocarbon groups directed outwardly from the core, in contact with the oil or lipid bulk phase. The core typically contains a sufficient number of cations to provide the whole assembly with a neutral charge. Such emulsion droplets generally have a radius ranging from about 10×10⁻⁸ cm to about 30×10⁻⁸ cm. Because the typical cell wall has a hydrophobic core bounded by a film having a thickness of about 30×10⁻⁸ cm, a closed emulsion droplet may not form in such a film since the film is too thin to surround the droplet. It is hypothesized, however, that a short cylinder of di(2-ethylhexyl)sulfosuccinate anions may form instead, with the anionic sulfonate groups directed inwardly toward an aqueous core and their hydrocarbon groups directed outwardly toward the lipid of the cell wall, and with the 2 ends of the cylinder open. One of the open ends is directed outward and the other is directed into the cell. Such a structure would act as a conduit through which the quaternary ammonium cation could reach the interior of target cells.

It is additionally hypothesized that these emulsion droplets and/or cylinders also may form in the mucosa of the intestine, with the quaternary ammonium cation acting as the neutralizing cation, thereby promoting the absorption of the quaternary ammonium cation through the intestinal walls in a similar manner as in the walls of the target cells.

The Compositions of the Present Invention

The compositions of the present invention may generally comprise a quaternary ammonium cation and a facilitating anion as described below. It is important to note that any of the compositions of the present invention may also preferably comprise one or more of the following, alone or in any appropriate combination: a neutralizing agent, a buffering agent, and/or an anti-cholinergic agent. Particularly preferred compositions fall within one of the following categories:

(1) Compositions comprising a quaternary ammonium cation selected from the group consisting of 2-PAM, pyridostigmine and neostigmine; and a facilitating anion. Compositions comprising 2-PAM may or may not include an anticholinergic agent such as atropine (or any equivalent, such as scopolamine, homoatropine, or methylatropine);

(2) Compositions comprising a quaternary ammonium cation selected from the group consisting of 2-PAM, pyridostigmine and neostigmine; a facilitating anion, a neutralizing agent and preferably, a buffering agent. Compositions comprising 2-PAM may or may not include an anticholinergic agent such as atropine (or any equivalent, such as scopolamine, homoatropine, or methylatropine); and

(3) Compositions comprising a quaternary ammonium cation selected from the group consisting of 2-PAM, pyridostigmine and neostigmine and aspirin (i.e., “acetylsalicylic acid”). Compositions comprising 2-PAM may or may not include an anticholinergic agent such as atropine (or its equivalent, such as scopolamine, homoatropine, or methylatropine).

Kits of the Invention

The current invention includes the concept of a kit, wherein the quaternary ammonium cation of the invention may be provided as a salt of a non-facilitating anion (such as bromide or chloride) and the facilitating anion may be provided as a salt of a non-quaternary ammonium salt (such as sodium or potassium). The kit may include such items as neutralizing agents, buffering agents and/or anti-cholinergic agents.

The quaternary ammonium cation, the neutralizing agent, the buffering agent and/or the anticholinergic agent and the facilitating anion together are present in the kit in a therapeutically effective amount such that their combination is therapeutically effective after they are administered. The individual components (e.g., the quaternary ammonium cation, the neutralizing agent, buffering agents, anti-cholinergic, etc.) may be any convenient combination of compositions in any convenient formulation such as pills, syrups, or liquids, and may be administered in any appropriate manner, e.g., parenterally or orally. Such compositions may include one or more of the individual components of the invention and each kit may have multiple compositions in it.

For kits, it is important to note that those skilled in the art will appreciate that the order of application of the components in the kit need not be in any specific order. However, in a preferred administration of the components of a kit of the present invention, the preferred order of administration is to administer the neutralizing agent and the buffer first to adjust the pH of the stomach prior to the administration of the source of the quaternary ammonium cation. A facilitating anion may or may not be added along with the neutralizing agent and the buffer. For example, prior to the administration of a salt containing a quaternary ammonium cation and a facilitating anion of the invention, it would be preferred to administer a commercially available Alka Seltzer®, which generally contains a neutralizing agent, a citric buffer and aspirin.

Source of the 2-PAM Cation

The compositions and kits of this invention contain the 2-PAM cation in the form of a pharmaceutically acceptable material that comprises the 2-PAM cation itself (such as 2-PAM chloride). Thus, for example, when intended for oral administration, the pharmaceutically acceptable material should release the 2-PAM cation into the aqueous contents of the gastrointestinal tract where it combines with the preferred anion of the invention (which may or may not come from the dissolution of a salt of the composition of 2-PAM and the facilitating anion). Suitable materials include, for example, pharmaceutically acceptable salts of the 2-PAM cation, such as 2-PAM chloride. Suitable materials also include a 2-PAM salt of the facilitating anion, such as 2-PAM combined with any of the anions of the invention, such as alkylsulfonate, alkylsulfosuccinate, alkylphosphate, dialkylphosphate, dialkanoylphosphatidate, dialkylsulfosuccinate, salicylate, or alkylsulfate.

Source of the Pyridostigmine Cation

The compositions and kits of this invention contain the pyridostigmine cation in the form of a pharmaceutically acceptable material that comprises the pyridostigmine cation. Thus, for example, when intended for oral administration, the pharmaceutically acceptable material should release the pyridostigmine cation into the aqueous contents of the gastrointestinal tract where it combines with the preferred anion of the invention (which may or may not come from the dissolution of a salt of the composition of pyridostigmine and the facilitating anion). Suitable materials include, for example, pharmaceutically acceptable salts of the pyridostigmine cation, such as pyridostigmine bromide. Suitable materials also include the pyridostigmine salts of the facilitating anions, such as pyridostigmine combined with any of the anions of the invention, such as alkylsulfonate, alkylsulfosuccinate, alkylphosphate, dialkylphosphate, dialkanoylphosphatidate, dialkylsulfosuccinate, salicylate, or alkylsulfate.

Other Potentiating Agents

Further, it is contemplated that compounds other than pyridostigmine may be suitable potentiating agents for use in the present invention. In particular, it is believed that dialkyl carbamates of primary alcohols may generally be used as potentiating agents in the present invention. For example, pyridostigmine derivatizes the serine residue of acetylcholinesterase by transferring a dimethylaminocarbonyl group to the serine hydroxyl group to form a carbamate. Thus, it is believed that dialkyl carbamates of primary alcohols in general may be used as cholinergic agents to derivatize acetylcholinesterase. In a preferred embodiment, suitable potentiating agents generally correspond to the formula RₐOC(O)NR_{b}R_{c}, wherein Rₐ is alkyl or quaternary ammonium; and R_{b} and R_{c} are independently hydrogen, alkyl, alkynyl, or cycloalkyl. In an alternative, preferred embodiment, —NR_{b}R_{c} is replaced by a cyclic secondary amino group —N(CH₂)ₙ, wherein n is 6 or less.

Source of the Neostigmine Cation

The compositions and kits of this invention contain the neostigmine cation in the form of a pharmaceutically acceptable material that either comprises the neostigmine cation. Thus, for example, when intended for oral administration, the pharmaceutically acceptable material should release the neostigmine cation into the aqueous contents of the gastrointestinal tract where it combines with the preferred anion of the invention (which may or may not come from the dissolution of a salt of the composition of neostigmine and the facilitating anion). Suitable materials include, for example, pharmaceutically acceptable salts of the neostigmine cation, such as neostigmine bromide. Suitable materials also include the neostigmine salts of the facilitating anions, such as neostigmine combined with any of the anions of the invention, such as alkylsulfonate, alkylsulfosuccinate, alkylphosphate, dialkylphosphate, dialkanoylphosphatidate, dialkylsulfosuccinate, salicylate, or alkylsulfate.

Source of the Facilitating Anion

The compositions and kits of this invention may contain the facilitating anion in the form of a pharmaceutically acceptable material that either comprises the facilitating anion itself or is capable of forming the facilitating anion after being administered to the intended recipient (and, consequently, when a composition or kit is referred to herein as comprising a facilitating anion, it should be understood that the composition or kit may either comprise the facilitating anion itself or the composition or components of the kit should be capable of forming the facilitating anion after being administered to the intended recipient). It is preferred that the facilitating anion have a weak affinity for water and/or be weakly polar, and it is particularly preferred for the facilitating anion to be less hydrophilic than a chloride, bromide or tosylate anion. Such a facilitating anion, when ingested with the quaternary ammonium cation (either in the form of a compound containing a salt of the quaternary ammonium cation of interest and the facilitating anion, or separate salts of the quaternary ammonium cation and the facilitating anion), tends to form cation/facilitating-anion combinations capable of, for example, crossing the lipid phase boundary of the gastrointestinal tract and entering the bloodstream, and crossing the lipid barriers of the capillary membranes of the nerve cells. Preferably, the facilitating anion forms quaternary ammonium cation/facilitating-anion combinations having a neutral or substantially neutral charge. Such combinations are also more lipophilic (or less hydrophilic) than 2-PAM chloride, pyridostigmine bromide or neostigmine bromide. Facilitating anion-containing materials, which have been approved by the Food and Drug Administration for use in other medicines or foods, are generally most preferred.

It is particularly preferred for the facilitating anion to have at least one of the following features:

(1) The facilitating anion is the conjugate base of an acid having a pKₐ value of less than about 5, more preferably less than about 4, and still more preferably less than about 3. Where a neutralizing agent is not administered, it is preferred for the facilitating anion to be the conjugate base of an acid having a pKₐ value of less than about 1, more preferably less than about 0, and still more preferably less than about −1. Although the pKₐ values associated with suitable facilitating anions may be less than about −10, most suitable facilitating anions will have a pKₐ value within the range of from about 1 to about −10, preferably from about 1 to about −2.

(2) The facilitating anion has a well-distributed charge to reduce its hydrophilicity. A particularly preferred example of such an anion is the salicylate anion.

(3) Alternatively, the facilitating anion comprises at least one alkyl group that comprises at least 10 carbon atoms. A preferred example of such an anion is the dodecylsulfate anion.

(4) The facilitating anion has an organic/aqueous phase distribution constant (“K”) that is greater than the organic/aqueous phase distribution constant associated with the tosylate anion (i.e., greater than about 320). In a particularly preferred embodiment, the facilitating anion has a K value which is greater than about 500, more preferably greater than about 700, still more preferably greater than about 800, and still even more preferably greater than about 1000. Although the K values associated with suitable facilitating anions may be greater than about 106, the most suitable facilitating anions have a K value of from about 500 to about 106. To determine the K value for a particular anion, a small amount of methyltridecylammonium chloride (hereinafter “Q⁺Cl⁻”) and a small amount of the sodium or potassium salt of the anion (“M⁺X⁻”) are added to a mixture of water and 1-decanol. The mixture is allowed to separate, and the concentrations of the chloride ion and the anion in each phase are then measured. The K value is calculated using the formula: K=[X⁻, dec.] [Cl⁻, aq.]/[X⁻, aq.] [Cl⁻, dec.], wherein the quantities in brackets are concentrations. The K value for the salicylate anion, for example, is reported to be greater than 1000. A more extensive discussion of the procedure for determining K values can be found in, for example, C. J. Coetzee and H. Freisee, Anal. Chem., Vol. 41, Page 1128 (1969) (incorporated herein by reference).

Examples of suitable facilitating anions include, but are not limited to, the following:

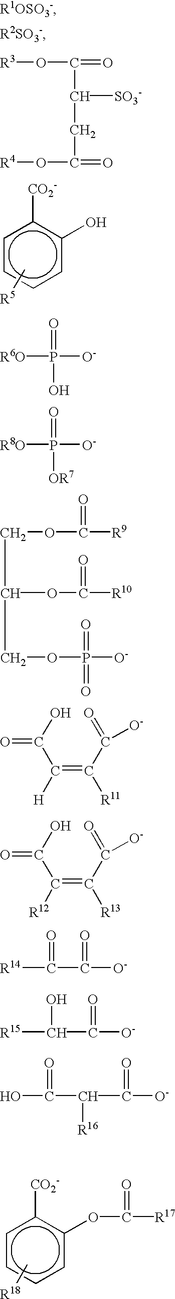

a pseudo-icosahedral carboranes anion (CB₁₁H₁₂⁻), and

a substituted pseudo-icosahedral carborane anion.

In the above-formulas, R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl. In a particularly preferred embodiment, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl; and R¹⁸ is hydrogen. In such an embodiment, R¹, R², R³, R⁵, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are preferably independently selected from the group consisting of alkyl, alkenyl, alkynyl, cycloalkyl, cycloalkenyl, aryl, arylalkyl, arylalkenyl, or arylalkynyl. The preferred aryl is phenyl. The aryl moiety may be unsubstituted or substituted with one or more radicals selected from the group consisting of alkyl, alkenyl, alkynyl, cycloalkyl, and cycloalkenyl.

In a preferred embodiment, R¹, R², R³, R⁴, R⁵, R⁶, R⁷, R⁸, R⁹, R¹⁰, R¹¹, R¹², R¹³, R¹⁴, R¹⁵, R¹⁶, R¹⁷ and the substituent (or substituents) of the substituted pseudo-icosahedral carborane anion are independently a residue of a fatty acid formed by removing a carboxylic acid group from the fatty acid.

In another preferred embodiment, the facilitating anion comprises an anion selected from the group consisting of (C₁₀-C₃₀)alkylsulfate anions, (C₁₀-C₃₀)alkylsulfonate anions, (C₆-C₁₂)alkylsulfosuccinate anions, salicylate anions, (C₁-C₃₀)alkylsalicylate anions, (C₁₀-C₃₀)alkylphosphate anions, di(C₈-C₁₂) alkylphosphate anions, di (C₁₀-C₃₀) alkanoylphosphatidate anions, (C₈-C₂₂)alkylmaleate anions, di(C₄-C₁₂)alkylmaleate anions, α-keto (C₉-C₂₁) carboxylate anions, α-hydroxy (C₉-C₂₁)carboxylate anions, (C₁₂-C₂₂)alkylmalonate anions, and (C₁-C₁₈)alkylpseudo-icosahedral carborane anions.

Still more preferably, the facilitating anion comprises an anion selected from the group consisting of (C₁₀-C₃₀)alkylsulfate anions, (C₁₀-C₃₀)alkylsulfonate anions, (C₆-C₁₂)alkylsulfosuccinate anions, salicylate anions, (C₁₀-C₃₀)alkylphosphate anions, di(C₈-C₁₂)alkylphosphate anions, and di(C₈-C₂₂) alkanoylphosphatidate anions.

Still even more preferred facilitating anions comprise an anion selected from the group consisting of the di(2-ethylhexyl)sulfosuccinate anion 2; the salicylate anion 3; the di(2-ethylhexyl) phosphate anion 4; the lauryl sulfate anion 5; the hexadecylsulfonate anion 6; the dipalmitoyl phosphatidate anion 7; and the acetylsalicylate anion 8:

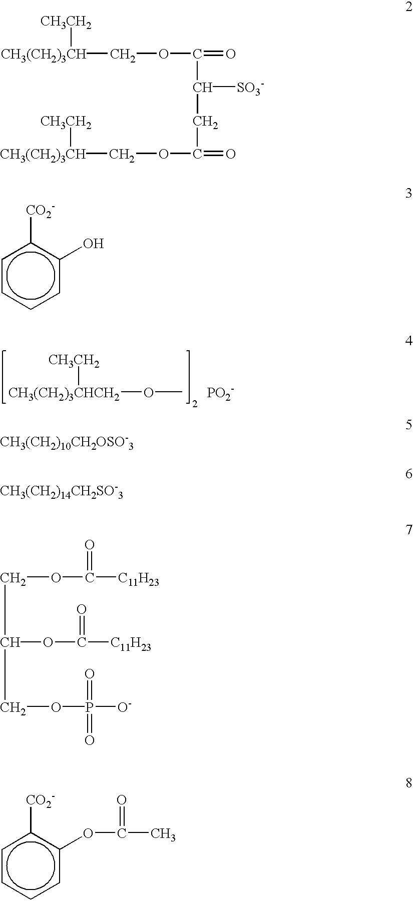

In a particularly preferred embodiment, the facilitating anion is the di(2-ethylhexyl)sulfosuccinate anion 2. In another particularly preferred embodiment, the facilitating anion is the salicylate anion 3. In yet another particularly preferred embodiment, the facilitating anion is the lauryl sulfate anion 5. In still a further particularly preferred embodiment, the facilitating anion is the acetylsalicylate anion 8. It is believed that these anions (and especially the salicylate anion, the lauryl sulfate anion, and the acetylsalicylate anion), in general, tend to synergistically enhance the therapeutic effects of the quaternary ammonium cation.

In one of the most preferred embodiments, the pharmaceutical composition or kit contains the acetylsalicylate anion in the form of acetylsalicylic acid (i.e., aspirin) or an alkali metal salt of acetylsalicylic acid (e.g., a sodium, potassium, calcium or magnesium salt of acetylsalicylic acid). Preferably, the acetylsalicylic acid or alkali metal salt of acetylsalicylic acid is given prior to or concomitantly with the administration of the quaternary ammonium cation. Although the acetylsalicylate anion may be provided in molar ratios under 1 (e.g., from about 0.5 to 1), it is preferred that at least a 1:1 molar ratio of acetylsalicylate anion to quaternary ammonium cation, most preferably a slight molar excess of acetylsalicylate (i.e., a molar ratio of acetylsalicylate anion to quaternary ammonium cation of from about 1.1 to about 1.5) should be utilized.

When intended for oral administration, the source of the facilitating anion preferably is a pharmaceutically acceptable material that releases the facilitating anion into the aqueous contents of the gastrointestinal tract. Non-limiting examples of suitable facilitating anion sources include the pharmaceutically acceptable salts of the facilitating anion (e.g., the alkali metal salts, particularly the sodium salts, of the facilitating anion), and solutions or suspensions comprising the facilitating anion. When the source is a salt, the counterion paired with the facilitating anion preferably has little or no tendency to associate with the facilitating anion. Such salts may be prepared by conventional means from the conjugate acid of the facilitating anion (e.g., reacting an appropriate base with the conjugate acid).

Sodium di(2-ethylhexyl)sulfosuccinate is commercially available from Aldrich Chemical Co., Milwaukee, Wis. Potassium di(2-ethylhexyl)sulfosuccinate can be prepared from the sodium salt by recrystallization from aqueous solution in the presence of an excess of potassium chloride.

Salicylic acid and sodium salicylate are both commercially available from Aldrich Chemical Co. Potassium salicylate can be prepared by treating a hot, concentrated solution of salicylic acid with an equivalent amount of potassium hydroxide, preferably as a concentrated solution, and then cooling to separate potassium salicylate.

Sodium dodecylsulfate is commercially available from Aldrich Chemical Co. Potassium dodecylsulfate can be prepared by recrystallizing the sodium salt in the presence of an excess of potassium chloride.

Di(2-ethylhexyl)phosphoric acid is commercially available from Aldrich Chemical Co. Sodium di(2-ethylhexyl)phosphate can be prepared by treating a toluene solution of the acid with a small excess of sodium hydroxide as an aqueous solution. A 2-phase system results, with the sodium salt in the toluene phase. Separation of the phases followed by distillation of the toluene yields a residue that is sodium di(2-ethylhexyl)phosphate. The potassium salt is obtained analogously, except that a potassium hydroxide solution is used in the place of the sodium hydroxide solution.

1-Hexadecylsulfonic acid sodium salt is commercially available from Aldrich Chemical Co. Potassium 1-hexadecylsulfonate can be obtained from the sodium salt by recrystallization in the presence of a small excess of potassium chloride.

Sodium salts of phosphatidic acids are commercially available from Avanti Polar Lipids, Alabaster, Alabama.

It should be recognized that the compositions, kits, and methods of the present invention are not limited to the use of a single type of facilitating anion. If necessary or desirable, 2 or more different types of facilitating anions can be used. It should also be recognized that the quaternary ammonium cation and the facilitating anion can be from the same compound or from different compounds. For example, the source of the quaternary ammonium cation and the source of the facilitating anion may be a single compound comprising the respective cation and the facilitating anion, such as a pharmaceutically acceptable salt wherein the respective cation is paired with the facilitating anion. Such compounds include, for example:

a) 2-PAM di(2-ethylhexyl)sulfosuccinate, pyridostigmine di(2-ethylhexyl)sulfosuccinate, and neostigmine di(2-ethylhexyl)sulfosuccinate;

b) 2-PAM salicylate, pyridostigmine salicylate, and neostigmine salicylate;

c) 2-PAM acetylsalicylate, pyridostigmine acetylsalicylate, and neostigmine acetylsalicylate;

d) 2-PAM di(2-ethylhexyl) phosphate, pyridostigmine di(2-ethylhexyl) phosphate, and neostigmine di(2-ethylhexyl) phosphate;

e) 2-PAM lauryl sulfate, pyridostigmine lauryl sulfate, and neostigmine lauryl sulfate; and

f) 2-PAM hexadecylsulfonate, pyridostigmine hexadecylsulfonate. and neostigmine hexadecylsulfonate.

Source of the Anti-Cholinergic

Anti-cholinergic agents such as atropine (or any equivalent, such as scopolamine, homoatropine, or methylatropine) may be applied with 2-PAM to prevent side reactions such as loss of blood pressure or excessive loss of heart rate. For example, atropine sulfate is benzeneacetic acid ((hydroxymethyl)-8-methy-8-azabicyclol [3.2.1] oct-3-yl ester, endo-+/−, sulfate (2:1) (salt), monohydrate). Atropine works as an anticholinergic by competitively inhibiting actions of acetylcholine at postganglionic parasympathetic neuroeffector sites. It is a competitive antagonist of acetylcholine at smooth and cardiac muscles and various glandular cells. Use of the an anticholinergic increases heart rate by slowing down some parts of the nervous system while simultaneously speeding up other parts. It relaxes bronchial smooth muscles, therefore reducing airway resistance and dead space.

Any source of commercially available, pharmaceutical grade atropine sulfate is acceptable. Equivalents to atropine may be used as well. Such equivalents include scopolamine, homoatropine, or methylatropine.

Common unit dosages for adult humans typically range from about 0.02 mg/kg to about 0.06 mg/kg (i.e., from about 2 mg to about 4 mg for a 70 kg adult). Generally, the anti-cholinergic may be applied every 5 minutes until signs of overdosage occur, e.g., tachycardia, excessive salivation or hypertension.

Source of the Neutralizing Agent

The compositions and kits of the present invention (particularly those intended to be orally administered) may optionally contain one or more neutralizing agents. The neutralizing agent may be any pharmaceutically acceptable material that increases the pH of the stomach when ingested, and that is chemically compatible with the quaternary ammonium cation and the facilitating anion selected. Preferably, the neutralizing agent is physiologically inert other than for pH adjustment purposes, and is not absorbed or only minimally absorbed from the gastrointestinal tract. Examples of particularly preferred neutralizing agents are those selected from the group consisting of pharmaceutically acceptable alkali metal carbonates (e.g., sodium bicarbonate or potassium hydrogen carbonate); alkali metal citrates (e.g., sodium citrate); alkali metal phosphates (e.g., disodium phosphate); alkali metal salts of carboxylic acids (e.g., alkali metal salts of acetic acid, tartaric acid or succinic acid); alkaline earth metal hydroxides (e.g., magnesium hydroxide); and mono-, di-, and polyamino-sugars (e.g., meglumine). In one of the more preferred embodiments, the neutralizing agent comprises sodium bicarbonate, sodium citrate, or a combination thereof, which are each non-toxic and have a lower equivalent weight than most other suitable neutralizing agents. For example, in a preferred embodiment, it has been found that a commercially available Alka Seltzer® tablet comprising sodium bicarbonate, citric acid and aspirin can act as a sufficient neutralizing agent.

A neutralizing agent permits the use of a broader class of facilitating anions. More specifically, because the preferred facilitating anions are conjugate bases of acids having a pKₐ value lower than or equal to the ambient pH of the stomach, a neutralizing agent can be used to temporarily increase the stomach pH in a subject to expand the range of suitable facilitating anions. When a neutralizing agent is employed, the facilitating anion selected preferably is the conjugate base of an acid having a pKₐ value at least about one unit less than the ambient pH as adjusted by the neutralizing agent, more preferably the conjugate base of an acid having a pKₐ value at least about 1.5 units less than the ambient pH as adjusted by the neutralizing agent, and still more preferably the conjugate base of an acid having a pKₐ value at least about 2 units less than the ambient pH as adjusted by the neutralizing agent.

Source of the Buffering Agent

The composition and kits of the present invention (particularly those intended for oral administration, and even more particularly those containing a neutralizing agent) may optionally contain one or more buffering agents to prevent an excessive increase in the pH of the aqueous contents of the stomach. The buffering agent may comprise any pharmaceutically acceptable buffering agent. Suitable buffering agents include, but are not limited to, pharmaceutically acceptable acids (e.g., citric acid). In a particularly preferred embodiment, the buffering agent is a pharmaceutically acceptable acid having a pKₐ value of at least about 1 unit (and more preferably at least about 2 units) greater than the pKₐ value of the conjugate acid of the facilitating anion selected. Even more preferably, the buffering agent is an acid having a pKₐ value of from about 4.5 to about 5.5.

Utility of the Compositions and Kits of the Present Invention

The 2-PAM compounds and kits of the invention are useful for the treatment of exposure to cholinesterase inhibitors, such as nerve gas (e.g., sarin, soman or VX nerve gases) or organophosphorus pesticides. Such compounds preferably include but are not limited to 2-PAM salicylate, 2-PAM lauryl sulfate and 2-PAM di(2-ethylhexyl sulfosuccinate). Although the body naturally clears cholinesterase inhibitors, the mechanism whereby it does so is too slow to prevent the afflicted individual from rapidly dying if a toxic amount of the inhibitor is absorbed. 2-PAM accelerates the clearance of the inhibitor, and it is critical to administer an antidote as soon as possible.

The current invention has the added benefit of being subject to administration either orally or parenterally. Oral dosages may provide for easier storage, availability and administration as opposed to parenteral application.

Pyridostigmine

The compounds and kits of the invention comprising potentiating agents, particularly those comprising pyridostigmine compounds and kits, are useful as an inhibitor of acetyl cholinesterase. The following pyridostigmine compounds are believed to be more preferred: pyridostigmine salicylate, pyridostigmine lauryl sulfate and pyridostigmine di(2-ethylhexyl sulfosuccinate).

The following theory of action, while believed to be accurate, is not intended to be binding. The potentiating agents esterify the crucial serine hydroxyl group of acetyl cholinesterase making a carbamate out of it (ROC(O)N(CH₃)₂). Sarin nerve gas is believed to work by derivatizing the same hydroxyl group, making phosphate esters instead of the carbamate. However the carbamate is easier to remove. When a potentiating agent such as pyridostigmine is administered, for example, prior to a threatened nerve gas attack, the subjects are given small, repeated doses of the drug, which results in the accumulation of inactivated, derivatized acetylcholinesterase. If the subject is then exposed to a cholinesterase inhibitor or other organophosphorus compound such as an agricultural pesticide, the carbamate-derivatized enzyme is unaffected as it is inactive. The subject then may take a calibrated dose of 2-PAM sufficient to liberate the acetylcholinesterase which has been stored as the carbamate. The 2-PAM readily cleaves off the carbamate from the reservoir of inactive enzyme, liberating a more readily available supply of acetyl cholinesterase. This occurs faster and more reliably than the liberation of the enzyme from the phosphate derivatives.

Neostigmine

The neostigmine compounds and kits of the invention are useful for alleviating suffering from myasthenia gravis, pseudo-obstruction of the bowel, paralytic ileus and/or urinary retention. While neostigmine cannot cure myasthenia gravis, it does allow the muscles to function more, and gives the afflicted individual more strength. Similarly, paralytic ileus and/or urinary retention and pseudo-obstruction of the bowel are failures of the nervous system that control discharge of the bladder and bowel respectively. Such failures lead to the inability of the individual to urinate or defecate, which is quite uncomfortable and can lead to serious complications. Application of neostigmine provides relief by allowing the nervous system to control the appropriate muscles.

The following theory of how neostigmine works, while believed to be correct, is not intended to be binding. Myasthenia gravis is a disease of muscle weakness. Neostigmine is a carbamate, which can transfer its carbamate functionality to the serine hydroxyl group of acetyl cholinesterase. This transfer inactivates the enzyme. In appropriate doses (not enough to inactivate all the enzyme) the acetyl choline level would be raised, leading to stronger nervous impulses to the muscles. Additionally, pyridostigmine would have the same effect, and has also been used in the treatment of myasthenia gravis.

A. Dosages

2-PAM

The pharmaceutical compositions and kits of the present invention preferably contain the 2-PAM cation in an amount sufficient to administer from about 200 to about 5000 mg, more preferably from about 400 to about 2500 mg, and most preferably from about 800 to about 1250 mg, of the 2-PAM cation (as 2-PAM). When the source of the of the 2-PAM cation is 2-PAM chloride, the pharmaceutical composition or kit preferably contains from about 500 to about 10,000 mg of 2-PAM chloride.

It should be recognized that the preferred daily dose of the 2-PAM cation will depend on various factors. One such factor is the specific condition being treated, i.e., the amount of sarin nerve gas to which the patient is exposed, or alternatively, the amount of organophosphorus pesticide to which the field worker has been exposed. It also should be recognized that the amount of the unit dosage and the dosage regimen for treating a condition may vary widely and will depend on a variety of factors, including the age, weight, sex, and medical condition of the subject; the severity of the condition; and the route and frequency of administration.

Typically, 2-PAM is administered to a patient exposed to a cholinesterase inhibitor in the form of an immediate unit dose, more preferably in the form of about 4 to 5 immediate unit doses, to assure that enough 2-PAM is absorbed into the system. Generally, the immediate doses are followed by repeated unit dosages every two hours until all symptoms have abated. When administered orally, the daily dose may be administered in the form of a unit dose of a composition comprising the 2-PAM cation or as part of a kit comprising a source of the 2-PAM cation. Thus, a typical daily dose of 2-PAM may comprise up to as many as 30 unit dosages, preferably between about 10 and about 20 unit dosages. A unit dosage typically contains, for example, between about 2 mg to about 75 mg, preferably from about 4 mg to about 40 mg, more preferably from about 8 mg to about 20 mg of the 2-PAM cation per kg of the recipient's body weight. Therefore, a typical daily dosage may comprise as much as 2500 mg or more of 2-PAM cation per kg of the recipient's body weight per day.

The higher range dosages are preferred for acute exposures. A salt of a facilitating anion such as acetyl salicylate, salicylate or lauryl sulfate will also be included in the tablet. The dosage unit form may be selected to accommodate the desired frequency of administration used to achieve the specified daily dosage. Preferably, at least an equimolar amount of facilitating anion (i.e., a 1:1 molar ratio of facilitating anion to 2-PAM cation) should be provided. More preferably, the composition or kit provides for a small molar excess of facilitating anion (i.e., a molar ratio of facilitating anion to 2-PAM cation of from about 1.1 to about 2, most preferably from about 1.1 to about 1.5). It is important to note that a molar ratio of facilitating anion to 2-PAM cation as low as 0.5 may be used, although there would be less facilitating anion present to combine with the 2-PAM.

Preferably, prior to ingestion of the 2-PAM tablets, the victim may also take a tablet of sodium bicarbonate buffered with citric acid, in order to neutralize any excess stomach acidity, which interferes with the rate of absorption of the 2-PAM. Alternatively, such ingredients may be included with the 2-PAM tablets.

According to a further alternative, a composition comprising the 2-PAM cation and facilitating anion may be prepared in a liquid form, which may or may not incorporate a neutralizing agent or buffering agent within it. In liquid form, the entire or part of a dosage would be administered upon exposure. Such a liquid form may preferably include 2-PAM salicylate, 2-PAM lauryl sulfate, 2-PAM acetyl salicylate, 2-PAM in combinations with any of the above facilitating anions, or combinations thereof.

If 2-PAM is provided along with an anti-cholinergic agent, the anti-cholinergic agent dosage would be sufficient to prevent any cardiac side-effects, such as bradycardia and hypotension. Typically, the anti-cholinergic agent is administered to treat excess acetylcholine activity. Thus, suitable dosages of the anti-cholinergic agent may vary with acetylcholine hyperactivity, as those skilled in the art will recognize. Generally, anti-cholinergic agent dosages range from 0 to about 7 micrograms, more preferably from about 1.5 to about 6 micrograms, and most preferably from about 3 to about 4.5 micrograms per kg of the recipient's body weight. Preferably, the anti-cholinergic agent is provided separately from the 2-PAM cation so the anti-cholinergic administration may be discontinued separately from the 2-PAM cation should excess acetylcholine activity cease.

Pyridostigmine

The pharmaceutical compositions and kits of the present invention preferably contain the pyridostigmine cation in an amount sufficient to administer a unit dose of the pyridostigmine cation of from about 1 to about 75 mg, more preferably from about 10 to about 50 mg, and most preferably from about 20 to about 40 mg.

Typical daily dosages of pyridostigmine provide from about 0.01 mg to about 4 mg, preferably from about 0.1 to about 3 mg, most preferably from about 0.25 to about 2 mg of pyridostigmine per kg of the recipient's body weight per day. The daily dose of pyridostigmine is preferably administered in the form of from 1 to 4 unit doses (e.g., the pyridostigmine is administered from once a day to every six hours). More preferably, the pyridostigmine is administered in 2 to 3 unit doses (every 8 to 12 hours), most preferably every 8 hours in the form of a unit dose of a composition comprising the pyridostigmine cation or as part of a kit comprising a source of the pyridostigmine cation.

Preferably, at least an equimolar amount of facilitating anion (i.e., a 1:1 molar ratio of facilitating anion to pyridostigmine cation) should be provided. More preferably, the composition or kit provides for a small molar excess of facilitating anion (i.e., a molar ratio of facilitating anion to pyridostigmine cation of from about 1.1:1 to about 2:1, most preferably about 1.5:1). Less preferably, a molar ratio of facilitating anion to pyridostigmine cation of about 0.5:1 may be used, although there would be less facilitating anion present to combine with the pyridostigmine cation.

Generally, dosages of pyridostigmine should be sufficient to build up a reservoir of carbamate-derivatized acetyl cholinesterase. The neutralizing and buffering agents are preferably provided as well to buffer the pH of the stomach in a preferable range from about 2 to 7.

Neostigmine

The pharmaceutical compositions and kits of the present invention preferably contain the neostigmine cation in an amount sufficient to administer a unit dose of the neostigmine cation of from about 1 to about 20 mg, more preferably from about 2 to about 15 mg, and most preferably from about 5 to about 10 mg.

Typical daily dosages of neostigmine provide from about 0.01 mg to about 1.5 mg of neostigmine, preferably from about 0.1 to about 1.0 mg of neostigmine, and most preferably from about 0.25 to about 0.75 mg of neostigmine per kg of the recipient's body weight per day. The daily dose of neostigmine is preferably administered in the form of from 1 to 4 unit doses (e.g., the neostigmine is administered from once a day to every six hours). More preferably, the neostigmine is administered in 2 to 3 unit doses (every 8 to 12 hours), most preferably every 8 hours in the form of a unit dose of a composition comprising the neostigmine cation or as part of a kit comprising a source of the neostigmine cation.

Preferably, at least an equimolar amount of facilitating anion (i.e., a 1:1 molar ratio of facilitating anion to neostigmine cation) should be provided. More preferably, the composition or kit provides for a small molar excess of facilitating anion (i.e., a molar ratio of facilitating anion to neostigmine cation of from about 1.1:1 to about 2:1, most preferably about 1.5:1). Less preferably, a molar ratio of facilitating anion to neostigmine cation of about 0.5:1 may be used, although there would be less facilitating anion present to combine with the neostigmine cation. Neutralizing and/or buffering agents are preferably provided as well to buffer the pH of the stomach in a preferable range from about 2 to 7.

B. The Ratio of Facilitating Anion to Quaternary Ammonium Cation

The pharmaceutical compositions and kits of the present invention preferably have a molar ratio of the facilitating anion to the quaternary ammonium cation of at least about 1.0, more preferably at least about 1.1, still more preferably from about 1.1 to about 4, and still even more preferably from about 1.1 to about 2. It is important to note that greater molar excesses of facilitating anion may be used, especially with the administration of 2-PAM to treat an acute exposure to a cholinesterase inhibitor.

In a preferred embodiment, for example, when the facilitating anion is an acetylsalicylate anion, the molar ratio of facilitating anion to quaternary ammonium cation is at least about 1.0, and more preferably from about 1.1 to about 1.5. Lower molar ratios (i.e., from about 0.5 to about 1.0) may also be used, but provide less facilitating anion.

It is important to note that when the composition or kit of the present invention comprises more than one facilitating anion or source of facilitating anion, the total amount of all facilitating anions within the composition or kit should be sufficient to provide a molar ratio of facilitating anion to quaternary ammonium cation of from 0.5 to about 2.0, more preferably from about 0.7 to about 1.5, and most preferably from about 0.9 to about 1.1. Thus, although one facilitating anion may be administered in a lesser amount, the combination of facilitating anions is sufficient to provide a molar ratio of facilitating anion to quaternary ammonium cation within the above ranges. For example, in a particularly preferred embodiment, a quaternary ammonium cation is administered in combination with a sodium dodecylsulfate anion and an acetylsalicylate anion. Preferably the combination is administered in the form of a composition comprising the sodium dodecylsulfate salt of the quaternary ammonium cation and an aspirin, and more preferably, the combination is administered as the components of a pharmaceutical kit further comprising a commercially available Alka Seltzer® tablet.

C. Neutralizing Agent

As noted above, the composition or kit preferably comprises a neutralizing agent when the composition or kit is being administered orally. The neutralizing agent preferably is administered in an amount sufficient to increase the pH of the aqueous contents of the stomach after ingestion to a value sufficient to prevent the absorption of a significant fraction of the facilitating anion as its conjugate acid into the gastrointestinal mucosa. More preferably, the amount of neutralizing agent is sufficient to temporarily increase the pH of the aqueous contents of the stomach to at least about 2, more preferably to at least about 3, and still more preferably to at least about 4. In a particularly preferred embodiment, the pH increases to at least about 2 within less than about 1 minute, and remains greater than about 2 for at least about 15 minutes after administration of the neutralizing agent. Although an amount of neutralizing agent sufficient to increase the pH to a value greater than about 7 may be used, the amount preferably does not increase the pH to a value greater than about 7. For most neutralizing agents, an amount of up to about 50 mmole (preferably from about 0.05 to about 50 mmole) is sufficient to achieve the desired pH increase in an average-size human. For example, when using sodium bicarbonate as the neutralizing agent in an average-size human, the sodium bicarbonate preferably is administered in an amount of from about 0.1 to about 4200 mg, more preferably from about 5 to about 4200 mg, still more preferably from about 10 to about 4200 mg, and still even more preferably from about 1000 to about 4200 mg.

It should be recognized that the facilitating anion also may function to increase the pH of the stomach when the facilitating anion is converted into its corresponding conjugate acid. Accordingly, the amount of neutralizing agent required may generally be reduced by increasing the amount of the facilitating anion in the composition. In some embodiments, the preference for a separate neutralizing agent may be entirely eliminated by selection of an appropriate amount of a suitable facilitating anion.

In one embodiment, it is preferred to use sodium bicarbonate as a neutralizing agent in combination with citric acid as a buffering agent as described below and as found commercially, for example, in an Alka Seltzer® tablet. In such an embodiment, the molar ratio of sodium bicarbonate to citric acid is preferably between about 3:1 to about 5:1, with a 4:1 molar ratio most preferred. Thus, a preferred composition of the present invention includes about 1000 mg to about 2000 mg (e.g., about 1250 mg, 1500 mg or 1700 mg) sodium bicarbonate and about 600 mg to about 1200 mg (e.g., about 750 mg, 900 mg or about 1000 mg) sodium citrate.

D. Buffering Agents

As noted above, the compositions intended to be administered orally preferably comprise a buffering agent, particularly where a neutralizing agent is also administered. When a buffering agent is used in combination with a neutralizing agent, the molar ratio of buffering agent to neutralizing agent may vary widely. Preferably, the molar ratio of buffering agent to neutralizing agent is from about 0.25 to about 1.5, and more preferably about 1. Typically, the amount of buffering agent should be sufficient to buffer the pH of the stomach between about 2 and 7 upon administration of the neutralizing agent. These proportions can be controlled either by incorporating the buffering agent and neutralizing agent in the appropriate relative proportions in a single tablet, pill or elixir, or by separately including the neutralizing agent and buffering agent in a kit which includes instructions for administering the kit components in appropriate proportions and/or means to control or facilitate control of the respective amounts administered.

In a particularly preferred embodiment as described above using sodium bicarbonate as a neutralizing agent in combination with citric acid as a buffering agent, the molar ratio of sodium bicarbonate neutralizing agent to citric acid buffering agent ranges from about 3:1 to about 5:1, and is most preferably about 4:1. Such formulations typically include from about 1000 to about 2000 mg (e.g., about 1250 mg, 1500 mg or 1700 mg) sodium bicarbonate and from about 600 mg to about 1200 mg (e.g., about 750 mg, 900 mg or about 1000 mg) with about 1700 mg sodium bicarbonate and about 1000 mg citric acid comprising a preferred composition.

Methods of Use

In accordance with the present invention, it has been discovered that the amount of the quaternary ammonium cation absorbed and/or the rate of absorption of the quaternary ammonium cation from the gastrointestinal tract (particularly the intestine) into the blood (i.e., the blood plasma or otherwise) and from the blood into the target cells can generally be improved by administering the quaternary ammonium cation (in a pharmaceutically acceptable source of the quaternary ammonium cation) to a subject, along with at least one source of lipophilic or weakly hydrophilic anion (i.e., a facilitating anion), and, optionally: (i) one or more neutralizing agents (e.g., sodium bicarbonate or sodium citrate) capable of temporarily increasing the pH of the aqueous contents in the stomach, (ii) one or more buffering agents (e.g., citric acid), and/or (iii) an anti-cholinergic agent (e.g., atropine). Further in accordance with the present invention, it has been discovered that 2-PAM, pyridostigmine or neostigmine can be orally administered in a formulation which comprises the 2-PAM, pyridostigmine or neostigmine cation together with a facilitating anion that is more hydrophobic than the respective chloride or bromide anion in a standard formulation, e.g., 2-PAM chloride ion, pyridostigmine bromide or neostigmine bromide.

Oral administration of formulations containing 2-PAM and such facilitating anions are believed to be highly and rapidly effective for arresting the toxic action of neurotoxic agents such as sarin nerve gases and organophosphorus pesticides. Additionally, oral administration of pyridostigmine can act as a potentiating agent (e.g., for 2-PAM if given ahead of expected exposure to sarin nerve gas or similar agent). Similarly, the oral administration of formulations containing neostigmine and such facilitating anions are highly and rapidly effective at treatment of myasthenia gravis, pseudo bowel obstruction, paralytic ileus and/or urinary retention. It will be understood that, while particularly suitable and desirable for oral administration, the 2-PAM, pyridostigmine or neostigmine compositions of the invention may also be adapted for intramuscular, intravenous, subcutaceous or other conventional manner of administration. The combination of the quaternary ammonium cations such as 2-PAM, pyridostigmine or neostigmine with facilitating anions assists its passage across biological membranes and increases the bioavailability of the 2-PAM, pyridostigmine or neostigmine, thereby permitting it to be taken orally or used in lower doses.

Biological membranes are lipid in character, as are nerve fibers. The 2-PAM, pyridostigmine or neostigmine must reach the nerve sites in order to be effective, i.e., they must pass from the blood, across the nerve blood barrier to the nerve fibers. If ingested orally, it must pass through the lipid mucosa of the intestine to reach the blood stream, and from there, be distributed to the nerve fibers.

Several methods can be used to transfer the 2-PAM, pyridostigmine or the neostigmine. In accordance with the preferred method, 2-PAM chloride is formulated with a pharmaceutically acceptable alkali metal or alkaline earth salt of an anion much less hydrophilic than chloride. Likewise, pyridostigmine bromide and neostigmine bromide are formulated with a pharmaceutically acceptable alkali metal or alkaline earth salt of a much less hydrophilic anion. The salt is then placed in a preparation that allows for oral ingestion. Upon oral ingestion, the salts dissolve in the stomach and the facilitating anion of the invention allows for greater uptake in the small intestine.

In another method, a 2-PAM salt (or other quaternary ammonium salt) of the facilitating anion is prepared and administered to the victim of exposure to the cholinesterase inhibitor. Both methods utilize the facilitating anion to promote transfer of the 2-PAM cation across the lipid membrane and to the target tissue. Likewise, the a salt comprising the pyridostigmine cation and facilitating anion may be prepared and administered in anticipation of the need for administration of 2-PAM. Similarly, the a salt comprising the neostigmine cation and facilitating anion may be prepared and administered to treat a patient suffering from myasthenia gravis, pseudo bowel obstruction, paralytic ileus and/or urinary retention. Again, the facilitating anion helps to promote the transfer of the quaternary ammonium cation to the appropriate nerve site. These salts may be applied parenterally or using any conventional method.

In an alternative embodiment, it is contemplated that quaternary ammonium salts comprising a non-facilitating anion could be ingested along with a salt comprisng a cation other than a quaternary ammonium cation (e.g., sodium, potassium, calcium or magnesium) and a facilitating anion. These salts could be combined together in a composition or alternatively be made available in a kit. The quaternary ammonium cation and the facilitating anion associate in the stomach upon mixing to allow uptake in the small intestine.

It is further contemplated that any of the above methods (compounds or kits) could include the use of neutralizing agents, buffering agents and/or anticholinergic agents. Such reagents could be incorporated into the compound or added separately into a kit. Use of the neutralizing agents and buffers can increase the stomach pH to the range of 2-7, thereby allowing furthered uptake of the quaternary ammonium cation with the facilitating anion.

It should also be understood that the invention contemplates the administration of the quaternary ammonium cations and the facilitating anions through compositions which contain mixtures of salts, i.e., a salt of the quaternary ammonium cation and a non-facilitating anion mixed with a salt of a non-quaternary ammonium cation and a facilitating anion. Such a composition can be ingested and allow the ions of interest to combine within the stomach and small intestine. Such mixtures may optionally include the use of other components such as neutralizing agents, buffering agents, and/or anticholinergic agents.

Suitable facilitating anions have aqueous to organic partition coefficients substantially larger than that of chloride ion or bromide ion. Examples include alkyl sulfates, alkyl sulfonates, mono- and dialkyl phosphates, o-acyl salicylates, C-alkylsalicylates, and salicylate ion itself. The aqueous to organic partition coefficients measure the distribution ratio of the quaternary ammonium cation between an octanol-rich liquid phase and the aqueous-rich phase.

To measure the partition coefficient for a facilitating anion in the presence of a quaternary ammonium compound, an acidified aqueous solution of the quaternary ammonium compound with a non-facilitating anion (e.g., tosylate, bromide or chloride) is first prepared. Sodium bicarbonate and a salt comprising the facilitating anion to be tested are then added to the aqueous solution. The salt comprising the facilitating anion should be added in an amount sufficient to provide a 1:1 molar ratio of the facilitating anion to the quaternary ammonium cation. The sodium bicarbonate is provided in an amount sufficient to produce a preselected pH value. An equal volume of n-octanol is then added to this solution. The solution is shaken. The mixture is centrifuged to separate an octanol layer and an aqueous layer, and the distribution ratio of the quaternary ammonium cation between the octanol-rich phase and the aqueous-rich phase (that is, the partition coefficient) may be measured. This analytical approach is believed to provide a suitable model for evaluating the bioavailability of the quaternary ammonium cation when utilized with a facilitating anion.

A. Order of Administration of the Quaternary Ammonium Cation, the Facilitating Anion and Other Optional Ingredients of a Kit

As noted above, a kit may be used in accordance with this invention, wherein the therapeutic ingredients to be administered are contained in at least two separate, discrete sources. To illustrate, a source of the quaternary ammonium cation may be separate and discrete from a source of a facilitating anion. Or, to illustrate further, a source of a quaternary ammonium cation may also contain a facilitating anion, while a neutralizing agent is contained in a separate, discrete source. Or to illustrate even further, there may be two separate, discrete sources of ingredients which each contain a quaternary ammonium cation and a facilitating anion. Regardless, the use of a kit allows for the administration of two or more different ingredients independently of each other. This, in turn, permits, for example, more effective adjustment in the amount of the facilitating anion, neutralizing agent, buffering agent, and/or anti-cholinergic agent administered relative to the amount of the quaternary ammonium cation administered.

Typically, when a kit is used, it is preferred that the facilitating anion(s) and/or anticholinergic (for 2-PAM) (as well as any neutralizing agent and/or buffering agent) be administered jointly or within about 30 minutes before or after (and more preferably within about 15 minutes before or after) the quaternary ammonium cation is administered. An ingredient administered jointly with the quaternary ammonium cation may be administered as a component of a quaternary ammonium cation source (i.e., where the quaternary ammonium cation source is a composition containing the cation and the additional ingredient). Alternatively, the additional ingredient may be administered as a component of a source separate and distinct from the quaternary ammonium cation source (i.e., where the source of the additional ingredient is administered simultaneously with the cation source). Or, as another alternative, the source containing the additional ingredient may be combined with the quaternary ammonium cation source before the administration of the cation source, and thereby administered as a composition containing the quaternary ammonium cation and the additional ingredient.

In a particularly preferred embodiment, a kit is used which contains a source comprising a unit dosage of the quaternary ammonium cation and a separate source comprising a unit dosage of a facilitating anion. The kit may also contain one or more other ingredients (e.g., a neutralizing agent, a buffering agent, and/or atropine (or any equivalent, such as scopolamine, homoatropine, or methylatropine) (for 2-PAM)) which may be a component of the source of the quaternary ammonium cation, a component of the source of the facilitating anion, and/or a component of a source separate from the sources of the quaternary ammonium cation and facilitating anion.

In yet another particularly preferred embodiment, a source containing a unit dosage of a neutralizing agent (and, optionally, a unit dosage of a buffering agent) is initially administered. This is then followed by the administration of a source(s) containing the quaternary ammonium cation and a facilitating anion.

In still another particularly preferred embodiment, administration of a source(s) of the quaternary ammonium cation and a facilitating anion is followed (preferably immediately) by the administration of a source(s) containing a unit dosage of an anticholinergic such as atropine (or any equivalent, such as scopolamine, homoatropine, or methylatropine) (for 2-PAM).

B. Injectable Compositions

As noted above, many of the compositions and kits of the present invention may be administered parenterally. In one particularly preferred embodiment of this invention, an injectable composition is used which comprises the quaternary ammonium cation and a facilitating anion (e.g., the salicylate anion or the acetylsalicylate anion).

Other Quaternary Ammonium Cations

The pharmaceutical compositions and kits of the present invention also are useful for the oral administration of other nonpeptide cationic therapeutic agents, particularly therapeutic agents comprising quaternary ammonium cations, in accordance with the compositions and kits of the type discussed above. These pharmaceutical compositions and kits can be prepared as set forth in this application by replacing the 2-PAM, pyridostigmine or neostigmine cation with a comparable molar fraction of a cation of the desired cationic therapeutic agent, such as propyromazine.

2-PAM

For treatment of exposure to cholinesterase inhibitors, the compositions of the invention comprise 2-PAM and a facilitating anion, and may include one or more of the following: neutralizing agent(s), buffering agent(s). It may be given with or without an anticholinergic, such as atropine or its equivalent (such as scopolamine, homoatropine, or methylatropine).

The composition of the present invention falls with the following ranges: 1% to 60% 2-PAM cation and 1% to 60% facilitating anion; preferably, 1% to 90% salt of the 2-PAM cation and facilitating anion. Optionally and preferably, the following may also be present: 0.01% to 50% neutralizing agent; 0.01% to 30% buffering agent; and 0% to 1% anticholinergic agent.

Alternatively, this may be described as a 1.0 gm tablet, the tablet would be composed of the following composition: 1 to 900 mgs salt of the 2-PAM cation and facilitating anion. Optionally and preferably, the following may also be present: 0.01 to 500 mgs of neutralizing agent; 0.01 to 30 mgs of buffering agent; 0 to 10 mgs of anticholinergic agent; and a remainder of fillers, disintegrants, binding agents, adhesives, wetting agents, lubricants, glidants, anti-adherents, enteric coatings, inert diluents and/or surface active/dispersing agents. Any combination of the above is considered part of the invention.

Pyridostigmine

Pyridostigmine may be given as a potentiating agent for 2-PAM (pyridostigmine was applied to the troops of desert storm). The compositions of the invention comprise pyridostigmine and a facilitating anion, and may include one or more of the following: neutralizing agent(s), buffering agent(s).

The composition of the present invention comprises 1% to 60% pyridostigmine cation and 1% to 60% facilitating anion. Preferably, the composition of the present invention comprises 1% to 90% salt of the pyridostigmine cation and facilitating anion. Optionally and preferably, the composition may also comprise 0.01% to 60% neutralizing agent; and 0.01% to 35% buffering agent.

Alternatively, this may be described as a 0.1 gm tablet comprising 1 to 90 mgs salt of the pyridostigmine cation and facilitating anion, with the tablet optionally and preferably comprising 0.01 to 60 mgs of neutralizing agent; and 0.01 to 35 mgs of buffering agent with the remainder comprising fillers, disintegrants, binding agents, adhesives, wetting agents, lubricants, glidants, anti-adherents, enteric coatings, inert diluents and/or surface active/dispersing agents. Any combination of the above is considered part of the invention.

Neostigmine

For treatment of myasthenia gravis, pseudo-obstruction of the bowel, paralytic ileus and/or urinary retention, the compositions of the invention comprise neostigmine and a facilitating anion, and may include one or more neutralizing agents and/or buffering agents.

The composition of the present invention comprise 1% to 60% neostigmine cation and 1% to 60% facilitating anion. Preferably, the composition comprises 1% to 90% salt of the neostigmine cation and facilitating anion. Optionally and preferably, the composition may further comprise 0.01% to 50% neutralizing agent; and 0.01% to 30% buffering agent.

Alternatively, the composition of the present invention may be described as a 0.3 gm tablet comprising 0.3 to 250 mgs salt of the neostigmine cation and facilitating anion. Optionally and preferably, the tablet may further comprise 0.003 to 200 mgs of neutralizing agent; and 0.003 to 120 mgs of buffering agent with a remainder comprising fillers, disintegrants, binding agents, adhesives, wetting agents, lubricants, glidants, anti-adherents, enteric coatings, inert diluents and/or surface active/dispersing agents. Any combination of the above is considered part of the invention.

Forms of Pharmaceutical Compositions for the Quaternary Ammonium Cation and Facilitating Anion(s)

The pharmaceutical compositions of the present invention concerning quaternary ammonium compounds selected from the group of 2-PAM, pyridostigmine and neostigmine comprise (a) the quaternary ammonium cation, (b) a facilitating anion(s) and may be used with an anticholinergic such as atropine (or its equivalent, such as scopolamine, homoatropine, or methylatropine). The compositions may include neutralizing agents and/or buffers. They may also comprise one or more non-toxic, pharmaceutically-acceptable carriers, excipients, and/or adjuvants (collectively referred to herein as “carrier materials”). The pharmaceutical compositions of the present invention may be adapted for administration by any suitable route by selection of appropriate carrier materials and a dosage of the quaternary ammonium cation effective for the intended treatment.

The techniques used to prepare the pharmaceutical compositions of this invention vary widely, and include the well known techniques of pharmacy for admixing the components of a medicine composition. In general, the compositions are prepared by uniformly and intimately admixing the active compounds (in the form of, for example, powders) with or without a liquid or finely divided solid carrier, or both, and then, if necessary, encapsulating or shaping the product. For example, a tablet may be prepared by compressing or molding a powder or granules of the compound, optionally with one or more accessory ingredients. Compressed tablets may be prepared by compressing, in a suitable machine, the compound in a free-flowing form, such as a powder or granules optionally mixed with a binding agent, lubricant, inert diluent, and/or surface active/dispersing agent(s). Molded tablets can be made by molding, in a suitable machine, the powdered compound moistened with an inert liquid diluent.

In a particularly preferred embodiment, the composition is intended to be administered orally. In this instance, the carrier material(s) may be solid and/or liquid. Preferably, such a composition is formulated as a unit-dose composition, i.e., the pharmaceutical composition contains a desired specific amount of the quaternary ammonium cation and the facilitating anion, and is in the form of, for example, a tablet (with or without a coating), a hard or soft capsule, a lozenge, a cachet, a dispensable powder, granules, a suspension, an elixir, a liquid, or any other form reasonably adapted for oral administration. Liquid dosage forms for oral administration include, for example, pharmaceutically acceptable emulsions, solutions, suspensions, syrups, and elixirs containing inert diluents commonly used in the art, such as water. Such compositions may also comprise, for example, wetting agents; emulsifying and suspending agents; and sweetening, flavoring, and perfuming agents. An excellent source which discusses in detail methods for preparing oral compositions (both solid and liquid) is Pharmaceutical Dosage Forms: Tablets, Second Edition, Revised and Expanded, Vol. 1-3 (ed. by Lieberman, H. A., Lachman, L., & Schwartz, J. B., Marcel Dekker, Inc., 270 Madison Ave, New York, N.Y. 1989) and Pharmaceutical Dosage Forms: Disperse Systems, Vol. 1-2 (ed. by Lieberman, H. A., Rieger, M. M., & Banker, G. S., Marcel Dekker, Inc., 270 Madison Ave, New York, N.Y. 1989).

The following discussion describes some of the more typical types of carrier materials that may be used in accordance with this invention. It should be recognized, however, that other carrier materials (such as colorants, flavors, sweeteners, and preservatives) are known in the pharmaceutical art, and may be used in the preparation of the pharmaceutical compositions of the present invention.

A. Diluents

The pharmaceutical compositions of the present invention may optionally comprise one or more pharmaceutically-acceptable diluents. Examples of suitable diluents include, either individually or in combination: lactose USP; lactose USP, anyhydrous; lactose USP, spray dried; starch USP; directly compressible starch; mannitol USP; sorbitol; dextrose monohydrate; microcrystalline cellulose NF; dibasic calcium phosphate dihydrate NF; sucrose-based diluents; confectioner's sugar; monobasic calcium sulfate monohydrate; calcium sulfate dihydrate NF; calcium lactate trihydrate granular NF; dextrates, NF (e.g., Emdex); Celutab; dextrose (e.g., Cerelose); inositol; hydrolyzed cereal solids such as the Maltrons and Mor-Rex; amylose; Rexcel; powdered cellulose (e.g., Elcema); calcium carbonate; glycine; bentonite; polyvinylpyrrolidone; and the like.

B. Disintegrants

The pharmaceutical compositions of the present invention may optionally comprise one or more pharmaceutically-acceptable disintegrants, particularly for tablet formulations. Examples of suitable disintegrants include, either individually or in combination: starches; sodium starch glycolate; clays (such as Veegum HV); celluloses and various modifications of celluloses (such as purified cellulose, methylcellulose and sodium carboxymethylcellulose, and carboxymethylcellulose); alginates; pregelatinized corn starches (such as National 1551 and National 1550); Crospovidone, USP NF; gums (such as agar, guar, locust bean, Karaya, pectin, tragacanth); and the like.

C. Binding Agents and Adhesives

The pharmaceutical compositions of the present invention may optionally contain one or more binding agents or adhesives, particularly for tablet formulations. Such a binding agent or adhesive preferably imparts sufficient cohesion to the powders to allow for normal processing, such as sizing, lubrication, compression and packaging, while also allowing the tablet to disintegrate and the composition to dissolve upon ingestion. Examples of suitable binding agents and adhesives include, either individually or in combination: acacia; tragacanth; sucrose; gelatin; glucose; starch; cellulose materials (e.g., methylcellulose and sodium carboxymethylcellulose (e.g., Tylose)); alginic acid and salts of alginic acid; magnesium aluminum silicate; polyethylene glycol; guar gum; polysaccharide acids; bentonites; polyvinylpyrrolidone; polymethacrylates; hydroxypropylmethylcellulose (HPMC); hydroxypropylcellulose (Klucel); ethylcellulose (Ethocel); pregelatinized starch (e.g., National 1511 and Starch 1500); and the like.

D. Wetting Agents

The pharmaceutical compositions of the present invention may optionally contain one or more pharmaceutically-acceptable wetting agents. Such wetting agents preferably maintain the quaternary ammonium cation, and, where desired, other ingredients of the composition in suspension, and improve the relative bioavailability of the pharmaceutical composition. Examples of suitable wetting agents include, either individually or in combination: oleic acid; glyceryl monostearate; sorbitan mono-oleate; sorbitan monolaurate; triethanolamine oleate; polyoxyethylene sorbitan mono-oleate; polyoxyethylene sorbitan monolaurate; sodium oleate; sodium lauryl sulfate; and the like.

E. Lubricants

The pharmaceutical compositions of the present invention may optionally contain one or more pharmaceutically-acceptable lubricants. The lubricant preferably (1) imparts a surface to the composition (e.g., in the form of a tablet or capsule) that allows simple removal of the composition from a mold, and/or (2) increases the ability of the components of the composition to be mixed evenly and readily. Examples of suitable lubricants include, either individually or in combination: glyceryl behapate (Compritol 888); stearates (magnesium, calcium, sodium); stearic acid; hydrogenated vegetable oils (e.g., Sterotex); talc; waxes; Stearowet; boric acid; sodium benzoate and sodium acetate; sodium fumarate; sodium chloride; DL-Leucine; polyethylene glycols (e.g., Carbowax 4000 and Carbowax 6000); sodium oleate; sodium benzoate; sodium acetate; sodium lauryl sulfate; magnesium lauryl sulfate; and the like.

F. Anti-Adherent Agents and Glidants

The pharmaceutical compositions of the present invention optionally may comprise one or more anti-adherent agents and/or glidants. Examples of suitable anti-adherents and glidants include, either individually or in combination: talc, cornstarch, Cab-O-Sil, Syloid, DL-Leucine, sodium lauryl sulfate, metallic stearates, and the like.

G. Enteric Coatings

In a particularly preferred embodiment, the pharmaceutical composition is in an enteric form, i.e., the pharmaceutical composition comprises a coating which is resistant to degradation in the stomach, but will decompose in the intestinal tract. In such an instance, the pharmaceutical composition is typically in the form of a tablet or capsule. Enteric coating materials are well-known in the art. For example:

1. In U.S. Pat. No. 4,849,227, Cho describes enteric coatings containing: hydroxypropyl methylcellulose phthalate, polyethylene glycol-6000, and/or shellac.

2. In U.S. Pat. No. 5,814,336, Kelm et al. describe polymer enteric coatings having a thickness of at least about 250 μm, and containing a polyanionic polymer that is insoluble in water and aqueous solutions having a pH of less than about 5 to about 6.3. Examples of coating materials that Kelm et al. report to be suitable are cellulose acetate phthalate, cellulose acetate trimelliate, hydroxypropyl methylcellulose phthalate, hydroxypropyl methyl cellulose acetate succinate, polyvinyl acetate phthalate, poly(methacrylic acid, methyl methacrylate) 1:1, poly(methacrylic acid, ethyl acrylate) 1:1, and compatible mixtures thereof.

3. In U.S. Pat. No. 5,914,132, Kelm et al. disclose a multilayered polymer enteric coating to prevent the release of an active ingredient until near the junction between the small intestine and the colon (or while in the colon). This multilayered coating has (1) an outer layer which has a thickness of from about 20 to about 50 μm, and begins to dissolve at a pH of between about 6.8 and about 7.2; and (2) an inner layer which has a thickness of roughly from about 90 to about 300 μm, and begins to dissolve at a pH of between about 5 and 6.3. Examples of coating materials that Kelm et al. report to be suitable for the outer coating are poly(methacrylic acid, methyl methacrylate) 1:2, and mixtures of poly(methacrylic acid, methyl methacrylate) 1:1 and poly(methacrylic acid, methyl methacrylate) 1:2 in a ratio of about 1:10 to about 1:2. Examples of coating materials that Kelm et al. report to be suitable for the inner coating are the same as those described as being suitable coatings in U.S. Pat. No. 5,814,336.

4. In U.S. Pat. No. 5,733,575, Mehra et al. describe enteric coatings made of titanized polyvinyl acetate phthalate, polyvinyl acetate phthalate which has been jet milled, hydroxypropyl methylcellulose phthalate, hydroxypropyl methylcellulose acetate succinate, or cellulose acetate phthalate. See also, e.g., Shaffer et al., U.S. Pat. No. 4,147,768; Maruyama et al., U.S. Pat. No. 5,750,148; Kukubo et al., U.S. Pat. No. 5,776,501; and Gardner et al., U.S. Pat. No. 5,980,951.

H. Injectable Compositions

The compositions of this invention are generally not limited to being used orally. In general, they also may be administered by injection (intravenous, intramuscular, subcutaneous, or jet) if desired. Such injectable compositions may employ, for example, saline, dextrose, or water as a suitable carrier material. The pH of the composition may be adjusted, if necessary, with a suitable acid, base, or buffer. Suitable bulking, dispersing, wetting, or suspending agents (e.g., mannitol and polyethylene glycol (such as PEG 400)), may also be included in the composition. A suitable parenteral composition can also include eplerenone in injection vials. Aqueous solutions can be added to dissolve the composition before injection. The compositions of this invention may also be contained in pre-filled syringes for emergency use.

An excellent source which discusses in detail methods for preparing injectable compositions is Pharmaceutical Dosage Forms: Parenteral Medications, Vol. 1-2 (ed. by Avis, K. E., Lachman, L., & Lieberman, H. A., Marcel Dekker, Inc., 270 Madison Ave, New York, N.Y. 1989).

Preparation of Quaternary Ammonium Salts with Hydrophobic or Weakly Hydrophilic Anions

Further in accordance with the invention, an advantageous process is provided for producing a salt of a quaternary ammonium cation and a relatively hydrophobic anion, i.e., more hydrophobic than a halide ion. In this process, an aqueous solution of a mineral acid salt of the quaternary ammonium cation is mixed with a source of alkali metal or alkaline earth metal salt of said anion. The resulting mixture is then contacted with a substantially water-immiscible organic solvent, thereby transferring the relatively hydrophobic salt of the quaternary ammonium cation and the more hydrophobic anion to the solvent phase and producing an organic extract comprising the transferred salt. An alkaline or alkaline earth salt of the mineral acid remains in the raffinate phase.

Halide salts of 2-PAM, pyridostigmine and neostigmine are commercially available, specifically, the chloride salt of 2-PAM and the bromide salts of the other two quaternary cations.

Preparation of Salts of 2-PAM, Neostigmine and Pyridostigmine

This section describes the preparation of salts of the physiologically active cations, 2-PAM, pyridostigmine, and neostigmine with hydrophobic anions, starting with cation of interest, preferably in the chloride or bromide salt form. 2-PAM is available from Aldrich as the chloride (2-pyridinealdoxime methochloride). Pyridostigmine bromide (3-dimethylaminocarbonyloxy-N-methylpyridinium bromide) and neostigmine bromide (3-[N,N-dimethylcarbamoyloxy]-N,N,N-trimethylanilinium bromide) are both available from Sigma.

An aqueous solution of the mineral acid salt of the quaternary ammonium cation is initially prepared, preferably at a concentration of between about 3% and about 30% by weight, more preferably between about 10% and about 20%, with a 15% concentration being preferred. To this solution is added a salt of the hydrophobic anion, preferably an alkali metal salt, e.g., sodium dodecylsulfate, potassium acetylsalicylate, etc. Preferably, the salt of the hydrophobic anion is added in proportions ranging from a stoichiometric equivalent of the quaternary ammonium salt (i.e., a molar ratio of hydrophobic anion to quaternary ammonium cation of from about 0.9:1 to about 1:1) to a slight molar excess of hydrophobic anion (i.e., molar ratios of hydrophobic anion to quaternary ammonium cation of from about 1:1 to about 1.5:1, preferably 1.1:1 to about 1.2:1).

The resulting mixture is agitated to achieve a homogeneous dispersion. A water-immiscible organic solvent, preferably chloroform, is then mixed with the dispersion, typically in a volumetric ratio of solvent to dispersion of between about 0.2 and about 5, preferably 1. Water is preferably added also, e.g., in volumetric ratio to the added solvent between about 0.2 and about 5, preferably 1. Separation of the phases yields and organic extract containing the desired quaternary ammonium salt of the hydrophobic anion.

The partition coefficients for these products (having a hydrophobic anion and a quaternary ammonium cation) are estimated to be at least about 5.

The extract may then by dried by contact with a suitable, unreactive, solid dessicant, such as anhydrous silica. The remaining solvent is removed by distillation. Chloroform, for example, may be removed under a moderate vacuum at about 40 [symbol] C. When distillation becomes quite slow, the pressure may be sufficiently lowered (e.g., to a few torr) to permit the remaining solvent to be removed. The solvent and distillation pressure are preferably selected to maintain the bottoms temperature below 50 [symbol] C in order to protect the stability of the product salt. Cooling the residue after evaporation of solvent yields a noncrystalline solid or a viscous liquid. This residue is the product salt. Its identity may be confirmed by near UV and/or IR spectroscopy. Typically, the product spectra will closely resemble a sum of the separately obtained spectra of the halide salts of the cations and the alkali metal salts of the anions, since these monatomic counterions should make no contributions to the spectra.

Definitions

The term “hydrocarbyl” refers to a group composed of carbon and hydrogen. This definition includes alkyl, alkenyl, and alkynyl groups which are each straight chain, branched chain, or cyclic hydrocarbons typically having from 1 to about 30 carbons atoms. Also included in this definition are aryl groups composed of carbon and hydrogen. Hydrocarbyl therefore includes, for example, methyl, ethyl, propyl, butyl, pentyl, hexyl, cyclopropyl, cyclobutyl, cyclopentyl, cyclohexyl, methylcyclopentyl, ethenyl, propenyl, butenyl, pentenyl, hexenyl, ethyne, propyne, butyne, pentyne, hexyne, phenyl, naphthyl, anthracenyl, benzyl, and isomers thereof.

The term “substituted hydrocarbyl” refers to a hydrocarbyl group in which one or more hydrogen has been substituted with a heteroatom-containing group. Such substituent groups include, for example, halo, oxo, heterocycle, alkoxy, hydroxy, aryloxy, —NO₂, amino, alkylamino, or amido. When the substituent group is oxo, the substituted hydrocarbyl can be, for example, an acyl group.

The term “alkyl” refers to linear or branched hydrocarbon groups having from 1 to about 30 carbon atoms. Examples of such groups include methyl, ethyl, n-propyl, isopropyl, n-butyl, isobutyl, sec-butyl, tert-butyl, pentyl, iso-amyl, hexyl, dodecyl, and the like. It should be recognized that such a group may be, for example, a residue of a saturated fatty acid formed by removing the carboxylic acid group from the fatty acid. More preferred alkyl groups are alkyl groups comprising at least 6 carbon atoms.

The term “alkenyl”, embraces linear or branched hydrocarbon groups having at least one carbon-carbon double bond, and from 2 to about 30 carbon atoms. Examples of alkenyl groups include ethenyl, allyl, propenyl, butenyl, 4-methylbutenyl, and the like. The term “alkenyl” embraces groups having “cis” and “trans” orientations, or, alternatively, “E” and “Z” orientations. It should be recognized that such a group may be, for example, a residue of an unsaturated fatty acid (having one or more double carbon-carbon bonds) formed by removing the carboxylic acid group from the fatty acid. More preferred alkenyl groups are alkyl groups comprising at least 6 carbon atoms.

The term “alkynyl” refers to linear or branched hydrocarbon groups having at least 1 carbon-carbon triple bond, and from 2 to about 30 carbon atoms. Examples of alkynyl groups include propargyl, 1-propynyl, 2-propynyl, 1-butyne, 2-butynyl, 1-pentynyl, and the like. More preferred alkyl groups are alkynyl groups comprising at least 6 carbon atoms.

The term “cycloalkyl” refers to saturated carbocyclic hydrocarbon groups having 3 to about 30 carbon atoms. Examples of such groups include cyclopropyl, cyclobutyl, cyclopentyl, cyclohexyl, and the like. More preferred cycloalkyl groups are “lower cycloalkyl” groups having from 3 to about 8 carbon atoms.

The term “cycloalkenyl” refers to partially unsaturated carbocyclic hydrocarbon groups having from 3 to about 30 carbon atoms. Examples of such groups include cyclobutenyl, cyclopentenyl, cyclohexenyl, and the like. More preferred cycloalkenyl groups are “lower cycloalkenyl” groups having from 4 to about 8 carbon atoms.

The term “aryl” refers to aromatic groups such as phenyl, naphthyl, tetrahydronaphthyl, indanyl, and biphenyl. The preferred aryl is phenyl. Aryl moieties may also be substituted at a substitutable position with one or more substituents selected independently from alkyl, alkenyl, alkynyl, cycloalkyl, cycloalkenyl, and the like. The term “aryl, alone or in combination” refers to a carbocyclic aromatic system containing 1, 2, or 3 rings, wherein such rings may be attached together in a pendent manner or may be fused.

The term “arylalkyl” refers to aryl-substituted alkyl groups such as benzyl, diphenylmethyl, triphenylmethyl, phenylethyl, and diphenylethyl. The aryl in the aralkyl may be additionally substituted with one or more substituents selected independently from alkyl, alkenyl, alkynyl, cycloalkyl, and cycloalkenyl. The terms “arylalkenyl” and “arylalkynyl” are defined in a comparable manner.

The term “pharmaceutically acceptable” means being compatible with the other components of the composition or kit being administered, and not deleterious to the intended recipient of the composition or kit.

The term “pharmaceutically-acceptable salts” refers to salts such as alkali metal salts, and common salts of free acids or free bases. The nature of the salt is not critical, provided that it is pharmaceutically-acceptable. Suitable pharmaceutically-acceptable salts of the quaternary ammonium cation and/or facilitating anion may be prepared from an inorganic acid or an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclyl, carboxylic, and sulfonic classes of organic acids (e.g., formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric, and galacturonic acid). Suitable pharmaceutically-acceptable salts of these compounds include metallic salts and organic salts. More preferred metallic salts include, but are not limited to, appropriate alkali metal (group IA) salts, alkaline earth metal (group IIA) salts, and other physiologically acceptable metals. Such salts can be made from aluminum, calcium, lithium, magnesium, potassium, and sodium. Preferred organic salts can be made from amines and quaternary ammonium salts, including, in part, trimethylamine, diethylamine, N,N′-dibenzylethylenediamine, chloroprocaine, choline, diethanolamine, ethylenediamine, meglumine (N-methylglucamine), and procaine.

The term “ventricular fibrillation threshold” refers to the lowest current level that, when applied to the heart, causes sustained ventricular fibrillation.

The term “effective ventricular refractory period” refers to the period during which the heart cannot be stimulated to contract by a super threshold electrical stimulus.

The term “rate-corrected Q-T_{c} interval” refers to the interval between the Q wave and the T wave, corrected for heart rate.

The term “prevent” means to at least partially suppress the onset of a condition.

With reference to the use of the word(s) “comprise” or “comprises” or “comprising” in this entire specification (including the claims below), Applicants note that unless the context requires otherwise, those words are used on the basis and clear understanding that they are to be interpreted inclusively, rather than exclusively, and that Applicants intend each of those words to be so interpreted in construing this entire specification.

### EXAMPLES

The following examples are simply intended to further illustrate and explain the present invention. This invention, therefore, should not be limited to any of the details in these examples. The symbols and conventions used in these examples are consistent with those used in the contemporary pharmacological literature. The facilitating anions used in these examples are commercially available or may be prepared as discussed above.

### Example 1

### Preparation of 2-PAM Pharmaceutical Composition

A pharmaceutical composition suitable for oral administration is prepared having the following composition:

2-PAM cation -8-16%

Facilitating anion, 20-40%

Neutralizing and buffering agents, 44-72%

### Example 2

### Preparation of a 2-PAM Pharmaceutical Composition

A pharmaceutical composition suitable for oral administration is prepared having the following composition

2-PAM chloride, 172 mg

Sodium dodecylsulfate (or lauryl sulfate), 300 mg

Neutralizing and buffering agents, 500 mg

### Example 3

### Preparation of a 2-PAM Pharmaceutical Composition

A pharmaceutical composition suitable for oral administration is prepared having the following composition

2-PAM chloride, 172 mg

Sodium di(2-ethylhexyl)sulfosuccinate, 500 mg

Neutralizing and buffering agents, 500 mg

### Example 4

### Determination of Partition Coefficients

Several formulations of the present invention were tested using an n-octanol/aqueous buffer system to measure the partition coefficients for the 2-PAM cation in the presence of the various facilitating anions. The experiment consisted of preparing an acidified aqueous solution of 2-PAM chloride. Sodium bicarbonate and a salt comprising the facilitating anion to be tested was then added to the aqueous solution. The salt comprising the facilitating anion was added in an amount sufficient to provide a 1:1 molar ratio of the facilitating anion to the 2-PAM cation. The sodium bicarbonate was provided in an amount sufficient to produce one of 4 preselected pH values. An equal volume of n-octanol was then added to this solution and the solution was shaken. The mixture was centrifuged to separate an octanol layer and an aqueous layer, and the distribution ratio of the 2-PAM cation between the octanol-rich phase and the aqueous-rich phase (that is, the partition coefficient) was measured. This analytical approach provides a suitable model for evaluating the bioavailability of the quaternary ammonium cation in the compositions tested.

A. Preparation of Aqueous 2-PAM Chloride Solution

The aqueous 2-PAM chloride solutions used in the procedure were prepared in the following manner. An amount of one of the buffer solutions described below (3.0 ml when the most acidic buffer solution was used, and 4.0 ml when the other 3 buffer solutions were used) was transferred to a beaker or an erlenmeyer flask by pipette. To the buffer solution was added 50 mg of 2-PAM chloride per ml of buffer solution and an equimolar amount of the sodium salt of the facilitating anion. For example, where the facilitating anion tested was di(2-ethylhexyl)sulfosuccinate, 54 mg of sodium di(2-ethylhexyl) sulfosuccinate was added per ml of buffer solution. Where the facilitating anion tested was salicylate, 19 mg of sodium salicylate was added per ml of buffer solution. Test solutions comprising other facilitating agents were prepared in a similar manner.

B. Preparation of Sodium Bicarbonate Buffer Solutions

Each of the pharmaceutical compositions was tested using each of the following sodium bicarbonate buffer systems:

The first buffer solution was prepared by dissolving 1.0 g of sodium bicarbonate in 100 ml of 0.95M HCl. A pH meter equipped with a conventional glass electrode and a calomel reference electrode was used to measure the nominal pH of this solution and the other 3 buffer solutions. The nominal pH measured for the first solution was 0.8. Because the glass electrode probably is not able to respond adequately to such an acidic solution, it is likely that the actual pH of this solution was lower, perhaps slightly negative. The nominal pH of this solution, however, was reproducible.

The second buffer solution was prepared by dissolving 7.5 g of sodium bicarbonate in 100 ml of 0.95M HCl. This solution had a reproducible nominal pH of 2.2, slightly higher than the expected pH of about 2.0.

The third buffer solution was prepared by dissolving 8.0 g of sodium bicarbonate in 100 ml of 0.95M HCl. This solution had a nominal pH between about 5.0 and about 6.0. While the pH meter is reliable in this pH range, the pH was somewhat variable because the solution had minimal buffer capacity.

The 4th buffer solution was prepared by dissolving 10.0 g of sodium bicarbonate in 100 ml of 0.95M HCl. This solution had a reproducible nominal pH of 7.7 that likely is close to the actual pH of the solution.

The first buffer solution was intended to model the acidity of the aqueous contents of the human stomach. The other 3 buffer solutions were intended to model the aqueous contents of the stomach after administration of an amount of sodium bicarbonate to reduce the acidity of the stomach.

C. Facilitating Anions Tested

The sodium salts of the following facilitating anions were tested: di(2-ethylhexyl)phosphate, di(2-ethylhexyl)sulfosuccinate, lauryl sulfate, and salicylate. All these salts were commercially available from Aldrich Chemical, Milwaukee, Wis. and/or Ecolab, Inc., St. Paul Minn. Each of the 4 facilitating anions was tested in each of the 4 buffer systems.

D. Preparation and Equilibration of n-Octanol/Buffer Systems

A 1.0 ml aliquot of the formulated aqueous test solution was added to 1.0 ml of n-octanol (Aldrich Chemical Co., HPLC grade, 99% purity), and the mixture was shaken for 30 seconds. The n-octanol and aqueous phases, which are not miscible, were separated by centrifuging at about 3000 rpm for about 10 minutes or until clarification was achieved. The phases were physically separated and the 2-PAM concentration of each phase was determined as described below. This procedure was followed for each combination of facilitating anion and buffer system. In addition, a corresponding control test was carried out without a facilitating anion in each of the buffer systems.

E. Measurement of Partition Coefficient

The concentration of the 2-PAM cation in the separated n-octanol and aqueous phases was determined by spectrophotometry using the long wavelength absorption of the 2-PAM cation after equilibration and appropriate dilution for proper scale. The absorbance of the aqueous solution was measured at 294 nm, and that of the octanol solution was measured at 300 nm. These were found to be the wavelengths of maximum absorbance for the long wavelength absorption band in these solvents. The molar coefficient of absorbance (extinction coefficient) was determined in water, by measuring the absorbance of 2-PAM chloride solutions of known concentration. The absorption coefficient at lambda(max) in octanol was assumed to be the same as that at lambda(max) in water. The value determined and used was 12,100.

To test the assumption about the absorption coefficient in octanol, the sum of the amount of 2-PAM found in the two phases was compared with the amount of 2-PAM chloride originally introduced, which was determined by weight. The discrepency was approximately 6% in one case, and the agreement was exact in the other. These results support the validity of the methods used, including the absorption coefficient for the octanol phase. When the spectrum of a substance like 2-PAM in an organic solvent is compared with its spectrum in water, a small shift to longer wave-length is often observed for the long wave-length absorption band. There is usually not much change in the absorption coeffiecient if both are measured at the maximum absorption.

From solutions at pH=6, with aqueous 2-PAM concentration initially 0.046 M, the partition coefficient is 0.00076 without sodium dodecylsulfate; 0.79 with an initial aqueous sodium dodecylsulfate concentration of 0.046 M. That is, practically no 2-PAM is extracted without SDS, and almost half of it is extracted with SDS on an equimolar basis. Obviously, increasing the amount of SDS increases the partition coefficient.

The extraction from a strongly acidic solution (1 M HCl) provided lower partition coefficient values; the partition coefficient was measured to be around 0.01. This indicates that the preferred invention of using a buffering agent and a neutralizing agent to improve the amount of 2-PAM absorbed into the system. Alternatively, enteric coatings could be used to protect the 2-PAM formulation in the stomach until it reaches the small intestine.

### Example 5

### Summary of 2-PAM Chloride Extraction Results

Aqueous solutions of 2-PAM chloride were equilibrated with equal volumes of octanol. The final concentrations of 2-PAM in the aqueous phase and in the octanol phase were determined spectrophotometrically.

The pH of the aqueous phases was adjusted with phosphate buffer and small volumes of HCl before equilibration. Various amounts of sodium dodecylsulfate were added as a facilitating agent. In all cases the initial 2-PAM chloride concentration was 4.6×10⁻² M.

The ratio between the organic and aqueous concentration is shown below in Table 1.

TABLE 1

| | Sodium | |
|---|---|---|
| | dodecylsulfate | |
| pH | concentration | K_{o/a} |
| 6 | 0.0 | 7.6 × 10⁻⁴ |
| 0 (added one | 4.6 × 10⁻² | 7.4 × 10⁻³ |
| molar HCl) | | |
| 6 | 4.6 × 10⁻² | 7.9 × 10⁻¹ |
| 6 | 9.2 × 10⁻² | 1.3 |
| 6 | 1.8 × 10⁻¹ | 5.9 × 10⁻¹ |

As demonstrated above, the addition of sodium dodecylsulfate improved the organic/aqueous concentration ratio by a factor of more than 1000. However, the experiment also indicated that the optimal effect was not achieved unless the pH was above zero.

### Example 6

### Comparison of Pyridostigmine Bromide Extraction into Octanol with 2-PAM Chloride Extraction

This example compares the partition coefficients of 2-PAM Chloride and Pyridostigmine Bromide, both with and without the use of a facilitating agent.

The experiment comprised equilibrating aqueous solutions of 2-PAM Chloride and Pyridostigmine Bromide with octanol as described above in Examples 4 and 5. In some of the experiments, Sodium dodecylsulfate and Sodium di(2-ethylhexyl)sulfosuccinate were used as facilitating agents for comparison. Partition coefficients were measured as described in the above Examples 4 and 5. Results of the experiment are summarized in Tables 2 and 3 below.

TABLE 2
2-PAM Extraction into Octanol with and without Sodium
dodecylsulfate (SDS)

| | Sodium | |
|---|---|---|
| | dodecylsulfate | |
| pH | concentration | K_{o/a} |
| 6^{a} | 0.0 | 7.6 × 10⁻⁴ |
| 0 (added one | 4.6 × 10⁻² | 7.4 × 10⁻³ |
| molar HCl) | | |
| 6^{a} | 4.6 × 10⁻² | 7.9 × 10⁻¹ |
| 6^{a} | 9.2 × 10⁻² | 1.3 |
| 6^{a} | 1.8 × 10⁻¹ | 5.9 × 10⁻¹ |

^{a}pH adjusted with phosphate buffer

TABLE 3
Pyridostigmine Bromide Extraction into Octanol
Facilitated by SDS
or Sodium di(2-ethylhexyl)sulfosuccinate (DOSS)

| | | Initial | |
|---|---|---|---|
| | Facilitating | Facilitating | |
| pH | Agent | Agent Conc. | K_{o/a} |
| 6^{a} | none | 0 | 9.9 × 10⁻⁴ |
| 0 (adjusted | none | 0 | 1.9 × 10⁻³ |
| w/1M HCl) | | | |
| 0 (adjusted | SDS | 0.11 | 2.3 × 10⁻² |
| w/1M HCl) | | | |
| 6^{a} | SDS | 0.11 | 1.54 |
| 0 | DOSS | 0.11 | 2.0 × 10⁻² |
| 6^{a} | DOSS | 0.11 | 0.92 |

^{a}pH adjusted w/ phosphate buffer.

The above description of the preferred embodiments is intended only to acquaint others skilled in the art with the invention, its principles, and its practical application, so that others skilled in the art may adapt and apply the invention in its numerous forms, as may be best suited to the requirements of a particular use. The present invention, therefore, is not limited to the above embodiments, and may be variously modified.

All patent documents and other literature references cited in this specification are incorporated herein by reference.

We claim:

1. A pharmaceutical combination useful for treating exposure to a cholinesterase inhibitor, the pharmaceutical combination comprising:
a 2-PAM cation or a source of a 2-PAM cation; and
a facilitating anion or a source of a facilitating anion, wherein:
the facilitating anion is less hydrophilic than a chloride anion; and
the 2-PAM cation or the source of the 2-PAM cation and the facilitating anion or the source of the facilitating anion together are present in the pharmaceutical combination in a therapeutically effective amount.

2. A pharmaceutical combination as set forth in claim 1 wherein the combination comprises a pharmaceutical composition comprising:
a 2-PAM cation; and
a facilitating anion, wherein:
the facilitating anion is less hydrophilic than a chloride anion; and
the 2-PAM cation and the facilitating anion together are present in the pharmaceutical combination in a therapeutically effective amount.

3. A pharmaceutical combination as set forth in claim 2, wherein said combination further comprises an anti-cholinergic agent.

4. A pharmaceutical combination as set forth in claim 3, wherein said anti-cholinergic agent comprises atropine.

5. A pharmaceutical combination as set forth in claim 2, wherein said combination further comprises a neutralizing agent.

6. A pharmaceutical combination as set forth in claim 5, wherein said neutralizing agent comprises sodium bicarbonate or sodium citrate.

7. A pharmaceutical combination as set forth in claim 5, wherein said combination further comprises a buffering agent.

8. A pharmaceutical combination as set forth in claim 7, wherein said buffering agent comprises citric acid.

9. A pharmaceutical combination as set forth in claim 2, wherein said facilitating anion has an organic/aqueous phase distribution equilibrium constant of greater than about 320 when introduced into a mixture comprising water, 1-decanol, methyltridecylammonium chloride, and a methyltridecylammonium salt.

10. A pharmaceutical combination as set forth in claim 2 wherein the molar ratio of said facilitating anion to said 2-PAM cation is from about 0.5 to about 2.

11. A pharmaceutical combination as set forth in claim wherein the molar ratio of said facilitating anion to said 2-PAM cation is from about 1.0 to about 1.5.

12. A pharmaceutical combination as set forth in claim wherein the molar ratio of said facilitating anion to said 2-PAM cation is from about 1.0 to about 1.1.

13. A pharmaceutical combination as set forth in claim 2, wherein, when the combination is orally administered to a human, the 2-PAM cation is absorbed into the bloodstream from the gastrointestinal tract.

14. A pharmaceutical combination as set forth in claim 2, wherein said facilitating anion comprises an anion selected from the group consisting of alkylsulfate, alkylsulfonate, alkylsulfosuccinate, salicylate, alkylsalicylate, alkylphosphate, dialkylphosphate, and dialkanoylphosphatidate.

15. A pharmaceutical composition as set forth in claim 14, wherein said facilitating anion comprises di(2-ethylhexyl)sulfosuccinate, salicylate, di(2-ethylhexyl) phosphate, hexadecylsulfonate, or dipalmitoyl phosphatidate.

16. A pharmaceutical composition as set forth in claim 15, wherein said facilitating anion comprises salicylate.

17. A pharmaceutical combination as set forth in claim 32, wherein said combination comprises a compound which comprises both said 2-PAM cation and said facilitating anion.

18. A pharmaceutical combination as set forth in claim 2, wherein said combination comprises 2-PAM di(2-ethylhexyl)sulfosuccinate, 2-PAM salicylate, 2-PAM acetylsalicylate, 2-PAM lauryl sulfate, 2-PAM di(2-ethylhexyl) phosphate, or 2-PAM hexadecylsulfonate.

19. A pharmaceutical combination as set forth in claim 2, wherein said pharmaceutical combination comprises a pharmaceutical composition comprising at least 2 types of facilitating anions.

20. A pharmaceutical combination as set forth in claim 2, wherein said combination is in a form comprising a tablet or a capsule.

21. A pharmaceutical combination as set forth in claim 2, wherein said combination is in a form comprising a solution or suspension.

22. A pharmaceutical combination as set forth in claim 2, wherein said combination is in a form suitable for administering via injection.

23. A pharmaceutical combination as set forth in claim 22, wherein said combination is suitable for intravenous or intramuscular injection.

24. A pharmaceutical combination as set forth in claim 22 further comprising a buffer.

25. A pharmaceutical combination as set forth in claim 24 further comprising a bulking, dispersing, wetting or suspending agent.

26. A pharmaceutical combination as set forth in claim 2, wherein said combination comprises a pharmaceutical composition comprising:
0.5% to 60% 2-PAM cation, and
0.3% to 60% facilitating anion.

27. A pharmaceutical combination as set forth in claim 26, wherein said combination further comprises 0.02% to 99% of a neutralizing agent and/or buffering agent.

28. A pharmaceutical combination as set forth in claim 2, wherein said combination comprises a pharmaceutical composition comprising:
5% to 20% 2-PAM cation, and
15% to 45% facilitating anion.

29. A pharmaceutical combination as set forth in claim 28, wherein said combination further comprises 35% to 80% of a neutralizing agent and/or buffering agent.

30. A pharmaceutical combination as set forth in claims 1-2, wherein said combination comprises a pharmaceutical composition comprising:
10% to 15% 2-PAM cation, and
30% to 40% facilitating anion.

31. A pharmaceutical combination as set forth in claim 30, wherein said combination further comprises 45% to 60% of a neutralizing agent and/or buffering agent.

32. A pharmaceutical combination as set forth in claim 1 wherein the combination comprises a pharmaceutical kit comprising:
a source of a 2-PAM cation; and
a source of a facilitating anion, wherein:
the facilitating anion is less hydrophilic than a chloride anion; and
the source of the 2-PAM cation and the source of the facilitating anion together are present in the pharmaceutical kit in a therapeutically effective amount.

33. A pharmaceutical combination as set forth in claim 32, wherein said combination comprises a pharmaceutical kit comprising at least 2 separate unit dosages, said unit dosages independently comprising said 2-PAM cation and said facilitating anion.

34. A pharmaceutical combination as set forth in claim 32, wherein said combination further comprises a source of an anti-cholinergic agent, a neutralizing agent and/or a buffering agent.

35. A pharmaceutical combination as set forth in claim 34, wherein said combination comprises a pharmaceutical kit comprising at least three separate unit dosages, said unit dosages independently comprising the 2-PAM cation, the facilitating anion, and the anti-cholinergic agent, neutralizing agent and/or buffering agent.

36. A pharmaceutical combination as set forth in claim 32, wherein said pharmaceutical kit further comprises a source of a neutralizing agent and a source of a buffering agent.

37. A pharmaceutical combination as as set forth in claim 36 wherein said neutralizing agent comprises sodium bicarbonate or sodium citrate and said buffering agent comprises citric acid.

38. A pharmaceutical combination as set forth in claim 37 wherein said sources of said neutralizing agent and said buffering agent are in combination and comprise a commercially available Alka Seltzer® tablet.

39. A pharmaceutical combination as set forth in claim 32, wherein said pharmaceutical combination comprises a pharmaceutical kit comprising at least 2 types of facilitating anions.

40. A pharmaceutical combination as set forth in claim 1, wherein the pharmaceutical combination comprises:
a 2-PAM cation or a source of a 2-PAM cation; and
a facilitating anion or a source of a facilitating anion, wherein:
the facilitating anion is less hydrophilic than a chloride anion;
the 2-PAM cation or the source of the 2-PAM cation and the facilitating anion or the source of the facilitating anion are, in combination, suitable for oral ingestion;
the 2-PAM cation or the source of the 2-PAM cation and the facilitating anion or the source of the facilitating anion are capable of forming a mixture comprising a 2-PAM cation and a facilitating anion within the gastrointestinal tract of a subject upon ingestion by the subject; and
the 2-PAM cation or the source of the 2-PAM cation and the facilitating anion or the source of the facilitating anion together are present in the pharmaceutical combination in a therapeutically effective amount.

41. A pharmaceutical combination as set forth in claim 40 wherein the combination comprises a pharmaceutical composition comprising:
a 2-PAM cation; and
a facilitating anion, wherein:
the facilitating anion is less hydrophilic than a chloride anion;
the 2-PAM cation and the facilitating anion are, in combination, suitable for oral ingestion;
the 2-PAM cation and the facilitating anion are capable of forming a mixture comprising a 2-PAM cation and a facilitating anion within the gastrointestinal tract of a subject upon ingestion by the subject; and
the 2-PAM cation and the facilitating anion together are present in the pharmaceutical combination in a therapeutically effective amount.

42. A pharmaceutical combination as set forth in claim 41, wherein said combination further comprises an anti-cholinergic agent.

43. A pharmaceutical combination as set forth in claim 42, wherein said anti-cholinergic agent comprises atropine.

44. A pharmaceutical combination as set forth in claim 41, wherein said combination further comprises a neutralizing agent.

45. A pharmaceutical combination as set forth in claim 44, wherein said neutralizing agent comprises sodium bicarbonate or sodium citrate.

46. A pharmaceutical combination as set forth in claim 44, wherein said combination further comprises a buffering agent.

47. A pharmaceutical combination as set forth in claim 46, wherein said buffering agent comprises citric acid.

48. A pharmaceutical combination as set forth in claim 41, wherein said facilitating anion has an organic/aqueous phase distribution equilibrium constant of greater than about 320 when introduced into a mixture comprising water, 1-decanol, methyltridecylammonium chloride, and a methyltridecylammonium salt.

49. A pharmaceutical combination as set forth in claim 41 wherein the molar ratio of said facilitating anion to said 2-PAM cation is from about 0.5 to about 2.

50. A pharmaceutical combination as set forth in claim 49 wherein the molar ratio of said facilitating anion to said 2-PAM cation is from about 1.0 to about 1.5.

51. A pharmaceutical combination as set forth in claim 49 wherein the molar ratio of said facilitating anion to said 2-PAM cation is from about 1.0 to about 1.1.

52. A pharmaceutical combination as set forth in claim 41, wherein, when the combination is orally administered to a human, the 2-PAM cation is absorbed into the bloodstream from the gastrointestinal tract.

53. A pharmaceutical combination as set forth in claim 41, wherein said facilitating anion comprises an anion selected from the group consisting of alkylsulfate, alkylsulfonate, alkylsulfosuccinate, salicylate, alkylsalicylate, alkylphosphate, dialkylphosphate, and dialkanoylphosphatidate.

54. A pharmaceutical composition as set forth in claim 53, wherein said facilitating anion comprises di(2-ethylhexyl)sulfosuccinate, salicylate, di(2-ethylhexyl) phosphate, hexadecylsulfonate, or dipalmitoyl phosphatidate.

55. A pharmaceutical composition as set forth in claim 54, wherein said facilitating anion comprises salicylate.

56. A pharmaceutical combination as set forth in claim 41, wherein said combination comprises a compound which comprises both said 2-PAM cation and said facilitating anion.

57. A pharmaceutical combination as set forth in claim 41, wherein said combination comprises 2-PAM di(2-ethylhexyl)sulfosuccinate, 2-PAM salicylate, 2-PAM acetylsalicylate, 2-PAM lauryl sulfate, 2-PAM di(2-ethylhexyl) phosphate, or 2-PAM hexadecylsulfonate.

58. A pharmaceutical combination as set forth in claim 41, wherein said pharmaceutical combination comprises a pharmaceutical composition comprising at least 2 types of facilitating anions.

59. A pharmaceutical combination as set forth in claim 41, wherein said combination is in a form comprising a tablet or a capsule.

60. A pharmaceutical combination as set forth in claim 41, wherein said combination is in a form comprising a solution or suspension.

61. A pharmaceutical combination as set forth in claim 41, wherein said combination is in a form suitable for administering via injection.

62. A pharmaceutical combination as set forth in claim 61, wherein said combination is suitable for intravenous or intramuscular injection.

63. A pharmaceutical combination as set forth in claim 61 further comprising a buffer.

64. A pharmaceutical combination as set forth in claim 63 further comprising a bulking, dispersing, wetting or suspending agent.

65. A pharmaceutical combination as set forth in claim 41, wherein said combination comprises a pharmaceutical composition comprising:
0.5% to 60% 2-PAM cation, and
0.3% to 60% facilitating anion.

66. A pharmaceutical combination as set forth in claim 65, wherein said combination further comprises 0.02% to 99% of a neutralizing agent and/or buffering agent.

67. A pharmaceutical combination as set forth in claim 41, wherein said combination comprises a pharmaceutical composition comprising:
5% to 20% 2-PAM cation, and
15% to 45% facilitating anion.

68. A pharmaceutical combination as set forth in claim 67, wherein said combination further comprises 35% to 80% of a neutralizing agent and/or buffering agent.

69. A pharmaceutical combination as set forth in claim 41, wherein said combination comprises a pharmaceutical composition comprising:
10% to 15% 2-PAM cation, and
30% to 40% facilitating anion.

70. A pharmaceutical combination as set forth in claim 69, wherein said combination further comprises 45% to 60% of a neutralizing agent and/or buffering agent.

71. A pharmaceutical combination as set forth in claim 40 wherein the combination comprises a pharmaceutical kit comprising:
a source of a 2-PAM cation; and
a source of a facilitating anion, wherein:
the facilitating anion is less hydrophilic than a chloride anion;
the source of the 2-PAM cation and the source of the facilitating anion are, in combination, suitable for oral ingestion;
the source of the 2-PAM cation and the source of the facilitating anion are capable of forming a mixture comprising a 2-PAM cation and a facilitating anion within the gastrointestinal tract of a subject upon ingestion by the subject; and
the source of the 2-PAM cation and the source of the facilitating anion together are present in the pharmaceutical combination in a therapeutically effective amount.

72. A pharmaceutical combination as set forth in claim 71, wherein said combination comprises a pharmaceutical kit comprising at least 2 separate unit dosages, said unit dosages independently comprising said 2-PAM cation and said facilitating anion.

73. A pharmaceutical combination as set forth in claim 71, wherein said combination further comprises a source of an anti-cholinergic agent, a neutralizing agent and/or a buffering agent.

74. A pharmaceutical combination as set forth in claim 73, wherein said combination comprises a pharmaceutical kit comprising at least three separate unit dosages, said unit dosages independently comprising the 2-PAM cation, the facilitating anion, and the anti-cholinergic agent, neutralizing agent and/or buffering agent.

75. A pharmaceutical combination as set forth in claim 71, wherein said pharmaceutical kit further comprises a source of a neutralizing agent and a source of a buffering agent.

76. A pharmaceutical combination as set forth in claim 75 wherein said neutralizing agent comprises sodium bicarbonate or sodium citrate and said buffering agent comprises citric acid.

77. A pharmaceutical combination as set forth in claim 76 wherein said sources of said neutralizing agent and said buffering agent are in combination and comprise a commercially available Alka Seltzer® tablet.

78. A pharmaceutical combination as set forth in claim 71, wherein said pharmaceutical combination comprises a pharmaceutical kit comprising at least 2 types of facilitating anions.

79. A pharmaceutical combination useful for treating exposure to a cholinesterase inhibitor, the pharmaceutical combination comprising:
a 2-PAM cation or a source of a 2-PAM cation; and
an anion or a source of an anion, wherein:
the anion comprises an anion selected from the group consisting of: 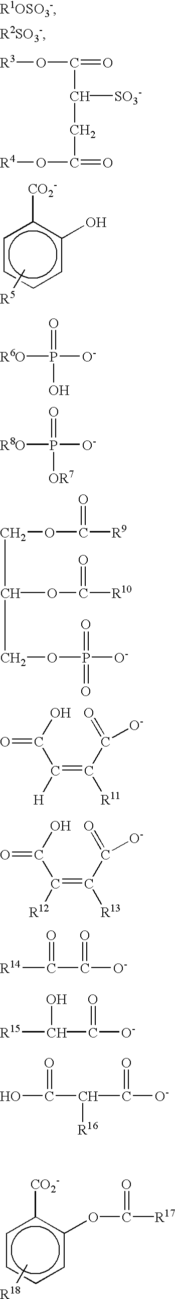
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

80. A pharmaceutical combination as set forth in claim 79 wherein the combination comprises a pharmaceutical composition comprising:
a 2-PAM cation; and
an anion comprising an anion selected from the group consisting of: 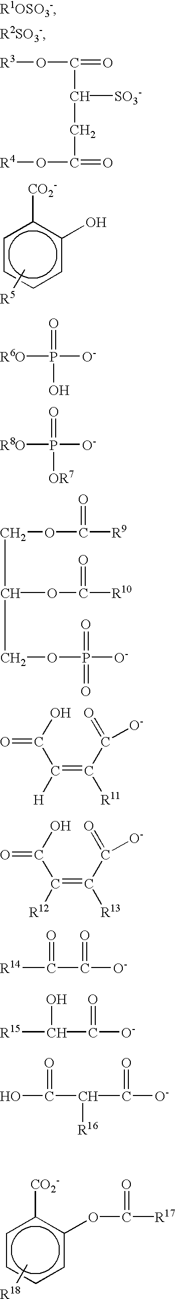
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

81. A pharmaceutical combination as set forth in claim 80, wherein the anion comprises R¹OSO₃⁻ and R¹ is hydrocarbyl or substituted hydrocarbyl.

82. A pharmaceutical combination as set forth in claim 81, wherein R¹ is hydrocarbyl.

83. A pharmaceutical combination as set forth in claim 80, wherein the anion comprises the formula: 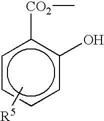
wherein R⁵ is hydrocarbyl or substituted hydrocarbyl.

84. A pharmaceutical combination as set forth in claim 83, wherein R¹ is hydrocarbyl.

85. A pharmaceutical combination as set forth in claim 80 wherein the anion comprises a compound having the formula: 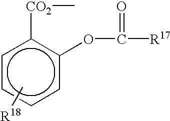
wherein:
R¹⁷ is hydrocarbyl or substituted hydrocarbyl; and
R¹⁸ is hydrogen, hydrocarbyl, or substituted hydrocarbyl.

86. A pharmaceutical combination as set forth in claim 85, wherein R¹⁷ is hydrocarbyl and R¹⁸ is hydrogen.

87. A pharmaceutical combination as set forth in claim 85, wherein the pharmaceutical combination comprises aspirin.

88. A pharmaceutical combination as set forth in claim 85, wherein the pharmaceutical composition is in a form suitable for administering orally.

89. A pharmaceutical combination as set forth in claim 79 wherein the combination comprises a pharmaceutical kit comprising:
a source of a 2-PAM cation; and
a source of an anion, wherein:
the anion comprises an anion selected from the group consisting of: 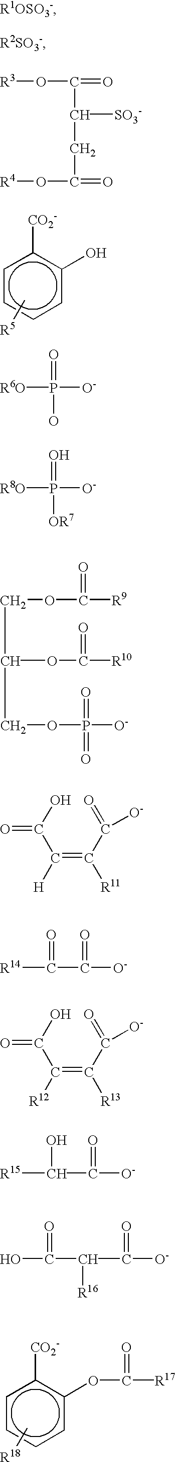
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

90. A pharmaceutical combination as set forth in claim 89, wherein the anion comprises R¹OSO₃⁻ and R¹ is hydrocarbyl or substituted hydrocarbyl.

91. A pharmaceutical combination as set forth in claim 90, wherein R¹ is hydrocarbyl.

92. A pharmaceutical combination as set forth in claim 89, wherein the anion comprises a compound having the formula: 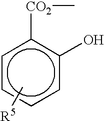
wherein R⁵ is hydrocarbyl or substituted hydrocarbyl.

93. A pharmaceutical combination as set forth in claim 92, wherein R¹ is hydrocarbyl.

94. A pharmaceutical combination as set forth in claim 89, wherein the anion comprises a compound having the formula: 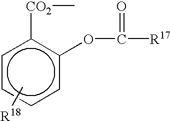
wherein:
R¹⁷ is hydrocarbyl or substituted hydrocarbyl; and
R¹⁸ is hydrogen, hydrocarbyl, or substituted hydrocarbyl.

95. A pharmaceutical combination as set forth in claim 94, wherein R¹⁷ is hydrocarbyl and R¹⁸ is hydrogen.

96. A pharmaceutical combination as set forth in claim 89, wherein the pharmaceutical kit comprises aspirin.

97. A pharmaceutical combination as set forth in claim 89, wherein the components of the pharmaceutical kit are each in a form suitable for administering orally.

98. A pharmaceutical combination useful for treating exposure to a cholinesterase inhibitor, the pharmaceutical combination comprising:
a 2-PAM cation or a source of a 2-PAM cation;
a facilitating anion or a source of a facilitating anion; and
an anticholinergic agent or a source of an anticholinergic agent, wherein:
the facilitating anion is less hydrophilic than a chloride anion;
the 2-PAM cation or the source of the 2-PAM cation and the facilitating anion or the source of the facilitating anion are capable of forming a mixture comprising a 2-PAM cation and a facilitating anion within the gastrointestinal tract of a subject upon ingestion by the subject; and
the 2-PAM cation or the source of the 2-PAM cation, the facilitating anion or the source of the facilitating anion and the anticholinergic agent or the source of the anticholinergic agent together are present in the pharmaceutical combination in a therapeutically effective amount.

99. A pharmaceutical combination as set forth in claim 98 wherein the combination comprises a pharmaceutical composition comprising:
a 2-PAM cation;
a facilitating anion; and
an anticholinergic agent, wherein:
the facilitating anion is less hydrophilic than a chloride anion;
the 2-PAM cation and the facilitating anion are, in combination, suitable for oral ingestion;
the 2-PAM cation and the facilitating anion are capable of forming a mixture comprising a 2-PAM cation and a facilitating anion within the gastrointestinal tract of a subject upon ingestion by the subject; and
the 2-PAM cation, the facilitating anion and the anticholinergic agent together are present in the pharmaceutical combination in a therapeutically effective amount.

100. A pharmaceutical combination as set forth in claim 99, wherein said anti-cholinergic agent comprises atropine.

101. A pharmaceutical combination as set forth in claim 99, wherein said combination further comprises a neutralizing agent.

102. A pharmaceutical combination as set forth in claim 101, wherein said neutralizing agent comprises sodium bicarbonate or sodium citrate.

103. A pharmaceutical combination as set forth in claim 101, wherein said combination further comprises a buffering agent.

104. A pharmaceutical combination as set forth in claim 103, wherein said buffering agent comprises citric acid.

105. A pharmaceutical combination as set forth in claim 99, wherein said facilitating anion has an organic/aqueous phase distribution equilibrium constant of greater than about 320 when introduced into a mixture comprising water, 1-decanol, methyltridecylammonium chloride, and a methyltridecylammonium salt.

106. A pharmaceutical combination as set forth in claim 99 wherein the molar ratio of said facilitating anion to said 2-PAM cation is from about 0.5 to about 2.

107. A pharmaceutical combination as set forth in claim 106 wherein the molar ratio of said facilitating anion to said 2-PAM cation is from about 1.0 to about 1.5.

108. A pharmaceutical combination as set forth in claim 106 wherein the molar ratio of said facilitating anion to said 2-PAM cation is from about 1.0 to about 1.1.

109. A pharmaceutical combination as set forth in claim 99, wherein, when the combination is orally administered to a human, the 2-PAM cation is absorbed into the bloodstream from the gastrointestinal tract.

110. A pharmaceutical combination as set forth in claim 99, wherein said facilitating anion comprises an anion selected from the group consisting of alkylsulfate, alkylsulfonate, alkylsulfosuccinate, salicylate, alkylsalicylate, alkylphosphate, dialkylphosphate, and dialkanoylphosphatidate.

111. A pharmaceutical composition as set forth in claim 110, wherein said facilitating anion comprises di(2-ethylhexyl)sulfosuccinate, salicylate, di(2-ethylhexyl) phosphate, hexadecylsulfonate, or dipalmitoyl phosphatidate.

112. A pharmaceutical composition as set forth in claim 111, wherein said facilitating anion comprises salicylate.

113. A pharmaceutical combination as set forth in claim 99, wherein said combination comprises a compound which comprises both said 2-PAM cation and said facilitating anion.

114. A pharmaceutical combination as set forth in claim 99, wherein said combination comprises 2-PAM di(2-ethylhexyl)sulfosuccinate, 2-PAM salicylate, 2-PAM acetylsalicylate, 2-PAM lauryl sulfate, 2-PAM di(2-ethylhexyl) phosphate, or 2-PAM hexadecylsulfonate.

115. A pharmaceutical combination as set forth in claim 99, wherein said pharmaceutical combination comprises a pharmaceutical composition comprising at least 2 types of facilitating anions.

116. A pharmaceutical combination as set forth in claim 99, wherein said combination is in a form comprising a tablet or a capsule.

117. A pharmaceutical combination as set forth in claim 99, wherein said combination is in a form comprising a solution or suspension.

118. A pharmaceutical combination as set forth in claim 99, wherein said combination is in a form suitable for administering via injection.

119. A pharmaceutical combination as set forth in claim 118, wherein said combination is suitable for intravenous or intramuscular injection.

120. A pharmaceutical combination as set forth in claim 118 further comprising a buffer.

121. A pharmaceutical combination as set forth in claim 120 further comprising a bulking, dispersing, wetting or suspending agent.

122. A pharmaceutical combination as set forth in claim 99, wherein said combination comprises a pharmaceutical composition comprising:
0.5% to 60% 2-PAM cation, and
0.3% to 60% facilitating anion.

123. A pharmaceutical combination as set forth in claim 122, wherein said combination further comprises 0.02% to 99% of a neutralizing agent and/or buffering agent.

124. A pharmaceutical combination as set forth in claim 99, wherein said combination comprises a pharmaceutical composition comprising:
5% to 20% 2-PAM cation, and
15% to 45% facilitating anion.

125. A pharmaceutical combination as set forth in claim 124, wherein said combination further comprises 35% to 80% of a neutralizing agent and/or buffering agent.

126. A pharmaceutical combination as set forth in claim 99, wherein said combination comprises a pharmaceutical composition comprising:
10% to 15% 2-PAM cation, and
30% to 40% facilitating anion.

127. A pharmaceutical combination as set forth in claim 126, wherein said combination further comprises 45% to 60% of a neutralizing agent and/or buffering agent.

128. A pharmaceutical combination as set forth in claim 98 wherein the combination comprises a pharmaceutical kit comprising:
a source of a 2-PAM cation;
a source of a facilitating anion; and
a source of an anticholinergic agent, wherein:
the facilitating anion is less hydrophilic than a chloride anion;
the source of the 2-PAM cation and the source of the facilitating anion are, in combination, suitable for oral ingestion;
the source of the 2-PAM cation and the source of the facilitating anion are capable of forming a mixture comprising a 2-PAM cation and a facilitating anion within the gastrointestinal tract of a subject upon ingestion by the subject; and
the source of the 2-PAM cation, the source of the facilitating anion and the source of the anticholinergic agent together are present in the pharmaceutical combination in a therapeutically effective amount.

129. A pharmaceutical combination as set forth in claim 128, wherein said combination comprises a pharmaceutical kit comprising at least 2 separate unit dosages, said unit dosages independently comprising said 2-PAM cation and said facilitating anion.

130. A pharmaceutical combination as set forth in claim 128, wherein said combination further comprises a source of a neutralizing agent and/or a buffering agent.

131. A pharmaceutical combination as set forth in claim 130, wherein said combination comprises a pharmaceutical kit comprising at least three separate unit dosages, said unit dosages independently comprising the 2-PAM cation, the facilitating anion, and the anti-cholinergic agent, neutralizing agent and/or buffering agent.

132. A pharmaceutical combination as set forth in claim 128, wherein said pharmaceutical kit further comprises a source of a neutralizing agent and a source of a buffering agent.

133. A pharmaceutical combination as as set forth in claim 132 wherein said neutralizing agent comprises sodium bicarbonate or sodium citrate and said buffering agent comprises citric acid.

134. A pharmaceutical combination as set forth in claim 133 wherein said sources of said neutralizing agent and said buffering agent are in combination and comprise a commercially available Alka Seltzer® tablet.

135. A pharmaceutical combination as set forth in claim 128, wherein said pharmaceutical combination comprises a pharmaceutical kit comprising at least 2 types of facilitating anions.

136. A method of treating exposure to a cholinesterase inhibitor, the method comprising administering a pharmaceutical combination of claim 1 to a subject in need thereof.

137. A method as set forth in claim 136 wherein said pharmaceutical combination comprises a pharmaceutical composition comprising:
a 2-PAM cation; and
a facilitating anion, wherein:
the facilitating anion is less hydrophilic than a chloride anion; and
the 2-PAM cation and the facilitating anion together are present in the pharmaceutical combination in a therapeutically effective amount.

138. A method as set forth in claim 136 wherein said pharmaceutical combination comprises a pharmaceutical kit comprising:
a source of a 2-PAM cation; and
a source of a facilitating anion, wherein:
the facilitating anion is less hydrophilic than a chloride anion; and
the source of the 2-PAM cation and the source of the facilitating anion together are present in the pharmaceutical kit in a therapeutically effective amount.

139. A method of treating exposure to a cholinesterase inhibitor, the method comprising administering a pharmaceutical combination of claim 40 to a subject in need thereof.

140. A method as set forth in claim 139 wherein said pharmaceutical combination comprises a pharmaceutical composition comprising:
a 2-PAM cation; and
a facilitating anion, wherein:
the facilitating anion is less hydrophilic than a chloride anion;
the 2-PAM cation and the facilitating anion are, in combination, suitable for oral ingestion;
the 2-PAM cation and the facilitating anion are capable of forming a mixture comprising a 2-PAM cation and a facilitating anion within the gastrointestinal tract of a subject upon ingestion by the subject; and
the 2-PAM cation and the facilitating anion together are present in the pharmaceutical combination in a therapeutically effective amount.

141. A method as set forth in claim 139 wherein said pharmaceutical combination comprises a pharmaceutical kit comprising:
a source of a 2-PAM cation; and
a source of a facilitating anion, wherein:
the facilitating anion is less hydrophilic than a chloride anion;
the source of the 2-PAM cation and the source of the facilitating anion are, in combination, suitable for oral ingestion;
the source of the 2-PAM cation and the source of the facilitating anion are capable of forming a mixture comprising a 2-PAM cation and a facilitating anion within the the source of the 2-PAM cation and the source of the facilitating anion are, in combination, suitable for oral ingestion;
the source of the 2-PAM cation and the source of the facilitating anion are capable of forming a mixture comprising a 2-PAM cation and a facilitating anion within the gastrointestinal tract of a subject upon ingestion by the subject; and
the source of the 2-PAM cation and the source of the facilitating anion together are present in the pharmaceutical combination in a therapeutically effective amount.

142. A method of treating exposure to a cholinesterase inhibitor, the method comprising administering a pharmaceutical combination of claim 79 to a subject in need thereof.

143. A method as set forth in claim 142 wherein said pharmaceutical combination comprises a pharmaceutical composition comprising:
a 2-PAM cation; and
a anion selected from the group consisting of: 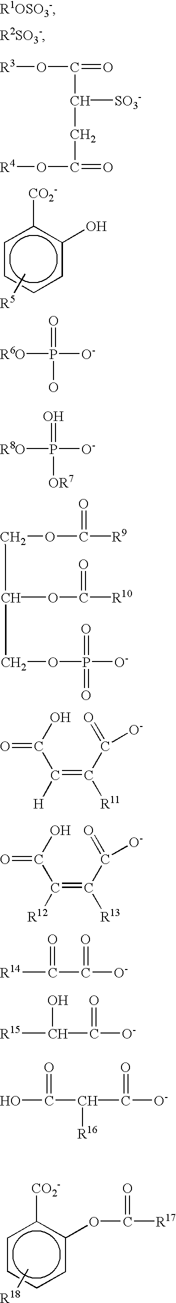
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

144. A method as set forth in claim 142 wherein said pharmaceutical combination comprises a pharmaceutical kit comprising:
a source of a 2-PAM cation; and
a source of a anion, wherein:
the anion comprises an anion selected from the group consisting of: 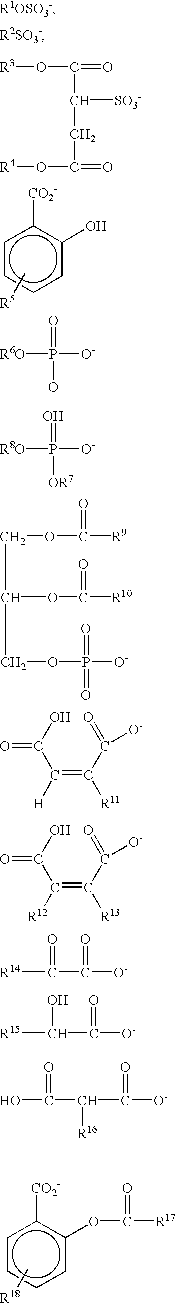
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

145. A method of treating exposure to a cholinesterase inhibitor, the method comprising administering a pharmaceutical combination of claim 98 to a subject in need thereof.

146. A method as set forth in claim 145 wherein said pharmaceutical combination comprises a pharmaceutical composition comprising:
a 2-PAM cation;
a facilitating anion; and
an anticholinergic agent, wherein:
the facilitating anion is less hydrophilic than a chloride anion;
the 2-PAM cation and the facilitating anion are, in combination, suitable for oral ingestion;
the 2-PAM cation and the facilitating anion are capable of forming a mixture comprising a 2-PAM cation and a facilitating anion within the gastrointestinal tract of a subject upon ingestion by the subject; and
the 2-PAM cation, the facilitating anion and the anticholinergic agent together are present in the pharmaceutical combination in a therapeutically effective amount.

147. A method as set forth in claim 145 wherein said pharmaceutical combination comprises a pharmaceutical kit comprising:
a source of a 2-PAM cation;
a source of a facilitating anion; and
a source of an anticholinergic agent, wherein:
the facilitating anion is less hydrophilic than a chloride anion;
the source of the 2-PAM cation and the source of the facilitating anion are, in combination, suitable for oral ingestion;
the source of the 2-PAM cation and the source of the facilitating anion are capable of forming a mixture comprising a 2-PAM cation and a facilitating anion within the gastrointestinal tract of a subject upon ingestion by the subject; and
the source of the 2-PAM cation, the source of the facilitating anion and the source of the anticholinergic agent together are present in the pharmaceutical combination in a therapeutically effective amount.

148. A pharmaceutical combination useful for potentiating clearance of a cholinesterase inhibitor, the pharmaceutical combination comprising:
a pyridostigmine cation or a source of a pyridostigmine cation; and
a facilitating anion or a source of a facilitating anion, wherein:
the facilitating anion is less hydrophilic than a bromide anion; and
the pyridostigmine cation or the source of the pyridostigmine cation and the facilitating anion or the source of the facilitating anion together are present in the pharmaceutical combination in a therapeutically effective amount.

149. A pharmaceutical combination as set forth in claim 148 wherein the combination comprises a pharmaceutical composition comprising:
a pyridostigmine cation; and
a facilitating anion, wherein:
the facilitating anion is less hydrophilic than a bromide anion; and
the pyridostigmine cation and the facilitating anion together are present in the pharmaceutical combination in a therapeutically effective amount.

150. A pharmaceutical combination as set forth in claim 149, wherein said combination further comprises a neutralizing agent.

151. A pharmaceutical combination as set forth in claim 150, wherein said neutralizing agent comprises sodium bicarbonate or sodium citrate.

152. A pharmaceutical combination as set forth in claim 150, wherein said combination further comprises a buffering agent.

153. A pharmaceutical combination as set forth in claim 152, wherein said buffering agent comprises citric acid.

154. A pharmaceutical combination as set forth in claim 149, wherein said facilitating anion has an organic/aqueous phase distribution equilibrium constant of greater than about 320 when introduced into a mixture comprising water, 1-decanol, methyltridecylammonium chloride, and a methyltridecylammonium salt.

155. A pharmaceutical combination as set forth in claim 149 wherein the molar ratio of said facilitating anion to said pyridostigmine cation is from about 0.5 to about 2.

156. A pharmaceutical combination as set forth in claim 155 wherein the molar ratio of said facilitating anion to said pyridostigmine cation is from about 1.0 to about 1.5.

157. A pharmaceutical combination as set forth in claim 155 wherein the molar ratio of said facilitating anion to said pyridostigmine cation is from about 1.0 to about 1.1.

158. A pharmaceutical combination as set forth in claim 149, wherein, when the combination is orally administered to a human, the pyridostigmine cation is absorbed into the bloodstream from the gastrointestinal tract.

159. A pharmaceutical combination as set forth in claim 149, wherein said facilitating anion comprises an anion selected from the group consisting of alkylsulfate, alkylsulfonate, alkylsulfosuccinate, salicylate, alkylsalicylate, alkylphosphate, dialkylphosphate, and dialkanoylphosphatidate.

160. A pharmaceutical composition as set forth in claim 159, wherein said facilitating anion comprises di(2-ethylhexyl)sulfosuccinate, salicylate, di(2-ethylhexyl) phosphate, hexadecylsulfonate, or dipalmitoyl phosphatidate.

161. A pharmaceutical composition as set forth in claim 160, wherein said facilitating anion comprises salicylate.

162. A pharmaceutical combination as set forth in claim 149, wherein said combination comprises a compound which comprises both said pyridostigmine cation and said facilitating anion.

163. A pharmaceutical combination as set forth in claim 149, wherein said combination comprises pyridostigmine di(2-ethylhexyl)sulfosuccinate, pyridostigmine salicylate, pyridostigmine acetylsalicylate, pyridostigmine lauryl sulfate, pyridostigmine di(2-ethylhexyl) phosphate, or pyridostigmine hexadecylsulfonate.

164. A pharmaceutical combination as set forth in claim 149, wherein said pharmaceutical combination comprises a pharmaceutical composition comprising at least 2 types of facilitating anions.

165. A pharmaceutical combination as set forth in claim 149, wherein said combination is in a form comprising a tablet or a capsule.

166. A pharmaceutical combination as set forth in claim 149, wherein said combination is in a form comprising a solution or suspension.

167. A pharmaceutical combination as set forth in claim 149, wherein said combination is in a form suitable for administering via injection.

168. A pharmaceutical combination as set forth in claim 167, wherein said combination is suitable for intravenous or intramuscular injection.

169. A pharmaceutical combination as set forth in claim 167 further comprising a buffer.

170. A pharmaceutical combination as set forth in claim 169 further comprising a bulking, dispersing, wetting or suspending agent.

171. A pharmaceutical combination as set forth in claim 149, wherein said combination comprises a pharmaceutical composition comprising:
1% to 60% pyridostigmine cation, and
1% to 60% facilitating anion.

172. A pharmaceutical combination as set forth in claim 171, wherein said composition further comprises 0.01% to 90% neutralizing agent and/or buffering agent.

173. A pharmaceutical combination as set forth in claim 148 wherein the combination comprises a pharmaceutical kit comprising:
a source of a pyridostigmine cation; and
a source of a facilitating anion, wherein:
the facilitating anion is less hydrophilic than a bromide anion; and
the source of the pyridostigmine cation and the source of the facilitating anion together are present in the pharmaceutical combination in a therapeutically effective amount.

174. A pharmaceutical combination as set forth in claim 173, wherein said combination comprises a pharmaceutical kit comprising at least 2 separate unit dosages, said unit dosages independently comprising said pyridostigmine cation and said facilitating anion.

175. A pharmaceutical combination as set forth in claim 173, wherein said combination further comprises a source of a neutralizing agent and/or a buffering agent.

176. A pharmaceutical combination as set forth in claim 175, wherein said combination comprises a pharmaceutical kit comprising at least three separate unit dosages, said unit dosages independently comprising the pyridostigmine cation, the facilitating anion, and the neutralizing agent and/or buffering agent.

177. A pharmaceutical combination as set forth in claim 175 wherein said neutralizing agent comprises sodium bicarbonate or sodium citrate and said buffering agent comprises citric acid.

178. A pharmaceutical combination as set forth in claim 177 wherein said sources of said neutralizing agent and said buffering agent are in combination and comprise a commercially available Alka Seltzer® tablet.

179. A pharmaceutical combination as set forth in claim 173, wherein said pharmaceutical combination comprises a pharmaceutical kit comprising at least 2 types of facilitating anions.

180. A pharmaceutical combination as set forth in claim 148, the pharmaceutical combination comprising:
a pyridostigmine cation or a source of a pyridostigmine cation; and
a facilitating anion or a source of a facilitating anion, wherein:
the facilitating anion is less hydrophilic than a bromide anion;
the pyridostigmine cation or the source of the pyridostigmine cation and the facilitating anion or the source of the facilitating anion are, in combination, suitable for oral ingestion;
the pyridostigmine cation or the source of the pyridostigmine cation and the facilitating anion or the source of the facilitating anion are capable of forming a mixture comprising a pyridostigmine cation and a facilitating anion within the gastrointestinal tract of a subject upon ingestion by the subject; and
the pyridostigmine cation or the source of the pyridostigmine cation and the facilitating anion or the source of the facilitating anion together are present in the pharmaceutical combination in a therapeutically effective amount.

181. A pharmaceutical combination as set forth in claim 180 wherein the combination comprises a pharmaceutical composition comprising:
a pyridostigmine cation; and
a facilitating anion, wherein:
the facilitating anion is less hydrophilic than a bromide anion;
the pyridostigmine cation and the facilitating anion are, in combination, suitable for oral ingestion;
the pyridostigmine cation and the facilitating anion are capable of forming a mixture comprising a pyridostigmine cation and a facilitating anion within the gastrointestinal tract of a subject upon ingestion by the subject; and
the pyridostigmine cation and the facilitating anion together are present in the pharmaceutical combination in a therapeutically effective amount.

182. A pharmaceutical combination as set forth in claim 181, wherein said combination further comprises a neutralizing agent.

183. A pharmaceutical combination as set forth in claim 182, wherein said neutralizing agent comprises sodium bicarbonate or sodium citrate.

184. A pharmaceutical combination as set forth in claim 182, wherein said combination further comprises a buffering agent.

185. A pharmaceutical combination as set forth in claim 184, wherein said buffering agent comprises citric acid.

186. A pharmaceutical combination as set forth in claim 181, wherein said facilitating anion has an organic/aqueous phase distribution equilibrium constant of greater than about 320 when introduced into a mixture comprising water, 1-decanol, methyltridecylammonium chloride, and a methyltridecylammonium salt.

187. A pharmaceutical combination as set forth in claim 181 wherein the molar ratio of said facilitating anion to said pyridostigmine cation is from about 0.5 to about 2.

188. A pharmaceutical combination as set forth in claim 187 wherein the molar ratio of said facilitating anion to said pyridostigmine cation is from about 1.0 to about 1.5.

189. A pharmaceutical combination as set forth in claim 187 wherein the molar ratio of said facilitating anion to said pyridostigmine cation is from about 1.0 to about 1.1.

190. A pharmaceutical combination as set forth in claim 181, wherein, when the combination is orally administered to a human, the pyridostigmine cation is absorbed into the bloodstream from the gastrointestinal tract.

191. A pharmaceutical combination as set forth in claim 181, wherein said facilitating anion comprises an anion selected from the group consisting of alkylsulfate, alkylsulfonate, alkylsulfosuccinate, salicylate, alkylsalicylate, alkylphosphate, dialkylphosphate, and dialkanoylphosphatidate.

192. A pharmaceutical composition as set forth in claim 191, wherein said facilitating anion comprises di(2-ethylhexyl)sulfosuccinate, salicylate, di(2-ethylhexyl) phosphate, hexadecylsulfonate, or dipalmitoyl phosphatidate.

193. A pharmaceutical composition as set forth in claim 192, wherein said facilitating anion comprises salicylate.

194. A pharmaceutical combination as set forth in claim 181, wherein said combination comprises a compound which comprises both said pyridostigmine cation and said facilitating anion.

195. A pharmaceutical combination as set forth in claim 181, wherein said combination comprises pyridostigmine di(2-ethylhexyl)sulfosuccinate, pyridostigmine salicylate, pyridostigmine acetylsalicylate, pyridostigmine lauryl sulfate, pyridostigmine di(2-ethylhexyl) phosphate, or pyridostigmine hexadecylsulfonate.

196. A pharmaceutical combination as set forth in claim 181, wherein said pharmaceutical combination comprises a pharmaceutical composition comprising at least 2 types of facilitating anions.

197. A pharmaceutical combination as set forth in claim 181, wherein said combination is in a form comprising a tablet or a capsule.

198. A pharmaceutical combination as set forth in claim 181, wherein said combination is in a form comprising a solution or suspension.

199. A pharmaceutical combination as set forth in claim 181, wherein said combination is in a form suitable for administering via injection.

200. A pharmaceutical combination as set forth in claim 199, wherein said combination is suitable for intravenous or intramuscular injection.

201. A pharmaceutical combination as set forth in claim 199 further comprising a buffer.

202. A pharmaceutical combination as set forth in claim 201 further comprising a bulking, dispersing, wetting or suspending agent.

203. A pharmaceutical combination as set forth in claim 181, wherein said combination comprises a pharmaceutical composition comprising:
1% to 60% pyridostigmine cation, and
1% to 60% facilitating anion.

204. A pharmaceutical combination as set forth in claim 203, wherein said composition further comprises 0.01% to 90% neutralizing agent and/or buffering agent.

205. A pharmaceutical combination as set forth in claim 180 wherein the combination comprises a pharmaceutical kit comprising:
a source of a pyridostigmine cation; and
a source of a facilitating anion, wherein:
the facilitating anion is less hydrophilic than a bromide anion;
the source of the pyridostigmine cation and the source of the facilitating anion are, in combination, suitable for oral ingestion;
the source of the pyridostigmine cation and the source of the facilitating anion are capable of forming a mixture comprising a pyridostigmine cation and a facilitating anion within the gastrointestinal tract of a subject upon ingestion by the subject; and
the source of the pyridostigmine cation and the source of the facilitating anion together are present in the pharmaceutical combination in a therapeutically effective amount.

206. A pharmaceutical combination as set forth in claim 204, wherein said combination comprises a pharmaceutical kit comprising at least 2 separate unit dosages, said unit dosages independently comprising said pyridostigmine cation and said facilitating anion.

207. A pharmaceutical combination as set forth in claim 204, wherein said combination further comprises a source of a neutralizing agent and/or a buffering agent.

208. A pharmaceutical combination as set forth in claim 207, wherein said combination comprises a pharmaceutical kit comprising at least three separate unit dosages, said unit dosages independently comprising the pyridostigmine cation, the facilitating anion, and the neutralizing agent and/or buffering agent.

209. A pharmaceutical combination as set forth in claim 207 wherein said neutralizing agent comprises sodium bicarbonate or sodium citrate and said buffering agent comprises citric acid.

210. A pharmaceutical combination as set forth in claim 209 wherein said sources of said neutralizing agent and said buffering agent are in combination and comprise a commercially available Alka Seltzer® tablet.

211. A pharmaceutical combination as set forth in claim 204, wherein said pharmaceutical combination comprises a pharmaceutical kit comprising at least 2 types of facilitating anions.

212. A pharmaceutical combination useful for potentiating clearance of a cholinesterase inhibitor, the pharmaceutical combination comprising:
a pyridostigmine cation or a source of a pyridostigmine cation; and
an anion or a source of an anion, wherein:
the anion comprises an anion selected from the group consisting of: 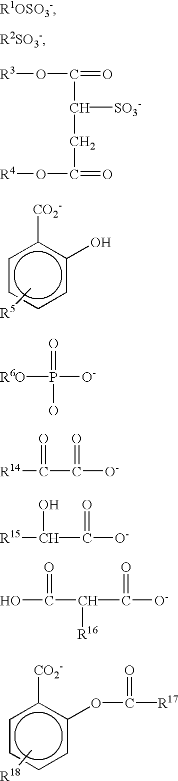
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

213. A pharmaceutical combination as set forth in claim 212 wherein the combination comprises a pharmaceutical composition comprising:
a pyridostigmine cation and an anion comprising an anion selected from the group consisting of: 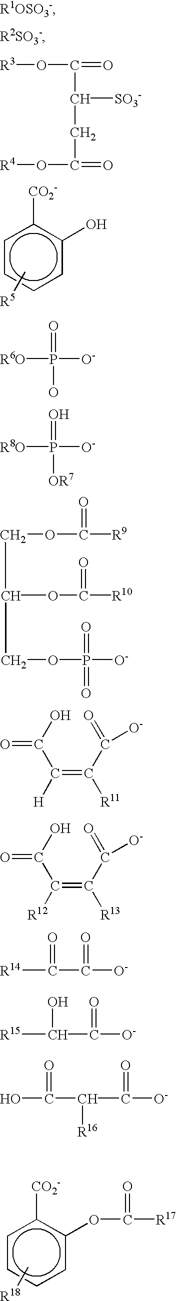
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

214. A pharmaceutical combination as set forth in claim 213, wherein the anion comprises R¹OSO₃⁻ and R¹ is hydrocarbyl or substituted hydrocarbyl.

215. A pharmaceutical combination as set forth in claim 214, wherein R¹ is hydrocarbyl.

216. A pharmaceutical combination as set forth in claim 213, wherein the anion comprises the formula: 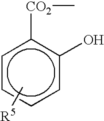
wherein R⁵ is hydrocarbyl or substituted hydrocarbyl.

217. A pharmaceutical combination as set forth in claim 216, wherein R⁵ is hydrocarbyl.

218. A pharmaceutical combination as set forth in claim 213 wherein the anion comprises a compound having the formula: 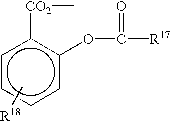
wherein:
R¹⁷ is hydrocarbyl or substituted hydrocarbyl; and
R¹⁸ is hydrogen, hydrocarbyl, or substituted hydrocarbyl.

219. A pharmaceutical combination as set forth in claim 218, wherein R¹⁷ is hydrocarbyl and R¹⁸ is hydrogen.

220. A pharmaceutical combination as set forth in claim 218, wherein the pharmaceutical combination comprises aspirin.

221. A pharmaceutical combination as set forth in claim 218, wherein the pharmaceutical composition is in a form suitable for administering orally.

222. A pharmaceutical combination as set forth in claim 212 wherein the combination comprises a pharmaceutical kit comprising:
a source of a pyridostigmine cation; and
a source of an anion, wherein:
the anion comprises an anion selected from the group consisting of: 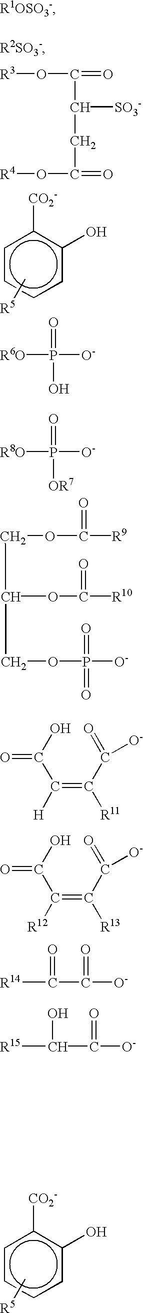
wherein R⁵ is hydrocarbyl or substituted hydrocarbyl.

226. A pharmaceutical combination as set forth in claim 92, wherein R⁵ is hydrocarbyl.

227. A pharmaceutical combination as set forth in claim 89, wherein the anion comprises a compound having the formula: 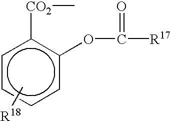
wherein:
R¹⁷ is hydrocarbyl or substituted hydrocarbyl; and
R¹⁸ is hydrogen, hydrocarbyl, or substituted hydrocarbyl.

228. A pharmaceutical combination as set forth in claim 227, wherein R¹⁷ is hydrocarbyl and R¹⁸ is hydrogen.

229. A pharmaceutical combination as set forth in claim 222, wherein the pharmaceutical kit comprises aspirin. 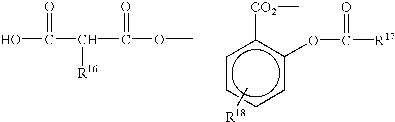
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

223. A pharmaceutical combination as set forth in claim 222, wherein the anion comprises R¹OSO₃⁻ and R¹ is hydrocarbyl or substituted hydrocarbyl.

224. A pharmaceutical combination as set forth in claim 223, wherein R¹ is hydrocarbyl.

225. A pharmaceutical combination as set forth in claim 222, wherein the anion comprises a compound having the formula:

230. A pharmaceutical combination as set forth in claim 222, wherein the components of the pharmaceutical kit are each in a form suitable for administering orally.

231. A pharmaceutical combination useful for potentiating clearance of a cholinesterase inhibitor, the pharmaceutical combination comprising:
a potentiating agent or a source of a potentiating agent; and
a facilitating anion or a source of a facilitating anion, wherein:
the facilitating anion is less hydrophilic than a chloride anion;
the potentiating agent or the source of the potentiating agent and the facilitating anion or the source of the facilitating anion are, in combination, suitable for oral ingestion;
the potentiating agent or the source of the potentiating agent and the facilitating anion or the source of the facilitating anion together are present in the pharmaceutical combination in a therapeutically effective amount; and
when the pharmaceutical combination is orally administered to a human, the potentiating agent is absorbed into the bloodstream from the gastrointestinal tract.

232. A pharmaceutical combination as set forth in claim 231 wherein said potentiating agent comprises a hydrophilic potentiating agent.

233. A pharmaceutical combination as set forth in claim 231 wherein said potentiating agent comprises a carbamate of a primary alcohol.

234. A pharmaceutical combination as set forth in claim 231 wherein said potentiating agent comprises a quaternary ammonium ion.

235. A pharmaceutical combination as set forth in claim 231 wherein said potentiating agent comprises pyridostigmine.

236. A pharmaceutical combination as set forth in claim 231, wherein the combination comprises a pharmaceutical composition comprising:
a potentiating agent; and
a facilitating anion, wherein:
the facilitating anion is less hydrophilic than a chloride anion; and
the potentiating agent and the facilitating anion together are present in the pharmaceutical combination in a therapeutically effective amount.

237. A pharmaceutical combination as set forth in claim 236, wherein said combination further comprises a neutralizing agent.

238. A pharmaceutical combination as set forth in claim 237, wherein said neutralizing agent comprises sodium bicarbonate or sodium citrate.

239. A pharmaceutical combination as set forth in claim 237, wherein said combination further comprises a buffering agent.

240. A pharmaceutical combination as set forth in claim 239, wherein said buffering agent comprises citric acid.

241. A pharmaceutical combination as set forth in claim 236, wherein said facilitating anion has an organic/aqueous phase distribution equilibrium constant of greater than about 320 when introduced into a mixture comprising water, 1-decanol, methyltridecylammonium chloride, and a methyltridecylammonium salt.

242. A pharmaceutical combination as set forth in claim 236 wherein the molar ratio of said facilitating anion to said potentiating agent is from about 0.5 to about 2.

243. A pharmaceutical combination as set forth in claim 242 wherein the molar ratio of said facilitating anion to said potentiating agent is from about 1.0 to about 1.5.

244. A pharmaceutical combination as set forth in claim 242 wherein the molar ratio of said facilitating anion to said potentiating agent is from about 1.0 to about 1.1.

245. A pharmaceutical combination as set forth in claim 236, wherein, when the combination is orally administered to a human, the potentiating agent is absorbed into the bloodstream from the gastrointestinal tract.

246. A pharmaceutical combination as set forth in claim 236, wherein said facilitating anion comprises an anion selected from the group consisting of alkylsulfate, alkylsulfonate, alkylsulfosuccinate, salicylate, alkylsalicylate, alkylphosphate, dialkylphosphate, and dialkanoylphosphatidate.

247. A pharmaceutical composition as set forth in claim 246, wherein said facilitating anion comprises di(2-ethylhexyl)sulfosuccinate, salicylate, di(2-ethylhexyl) phosphate, hexadecylsulfonate, or dipalmitoyl phosphatidate.

248. A pharmaceutical composition as set forth in claim 247, wherein said facilitating anion comprises salicylate.

249. A pharmaceutical combination as set forth in claim 236, wherein said combination comprises a compound which comprises both said potentiating agent and said facilitating anion.

250. A pharmaceutical combination as set forth in claim 236, wherein said pharmaceutical combination comprises a pharmaceutical composition comprising at least 2 types of facilitating anions.

251. A pharmaceutical combination as set forth in claim 236, wherein said combination is in a form comprising a tablet or a capsule.

252. A pharmaceutical combination as set forth in claim 236, wherein said combination is in a form comprising a solution or suspension.

253. A pharmaceutical combination as set forth in claim 236, wherein said combination is in a form suitable for administering via injection.

254. A pharmaceutical combination as set forth in claim 253, wherein said combination is suitable for intravenous or intramuscular injection.

255. A pharmaceutical combination as set forth in claim 253 further comprising a buffer.

256. A pharmaceutical combination as set forth in claim 255 further comprising a bulking, dispersing, wetting or suspending agent.

257. A pharmaceutical combination as set forth in claim 231, wherein the combination comprises a pharmaceutical kit comprising:
a source of a potentiating agent; and
a source of a facilitating anion, wherein:
the facilitating anion is less hydrophilic than a chloride anion; and
the source of the potentiating agent and the source of the facilitating anion together are present in the pharmaceutical combination in a therapeutically effective amount.

258. A pharmaceutical combination as set forth in claim 257, wherein said combination comprises a pharmaceutical kit comprising at least 2 separate unit dosages, said unit dosages independently comprising said potentiating agent and said facilitating anion.

259. A pharmaceutical combination as set forth in claim 257, wherein said combination further comprises a source of a neutralizing agent and/or a buffering agent.

260. A pharmaceutical combination as set forth in claim 259, wherein said combination comprises a pharmaceutical kit comprising at least three separate unit dosages, said unit dosages independently comprising the potentiating agnet, the facilitating anion, and the neutralizing agent and/or buffering agent.

261. A pharmaceutical combination as set forth in claim 259 wherein said neutralizing agent comprises sodium bicarbonate or sodium citrate and said buffering agent comprises citric acid.

262. A pharmaceutical combination as set forth in claim 261 wherein said sources of said neutralizing agent and said buffering agent are in combination and comprise a commercially available Alka Seltzer® tablet.

263. A pharmaceutical combination as set forth in claim 257, wherein said pharmaceutical combination comprises a pharmaceutical kit comprising at least 2 types of facilitating anions.

264. A method for potentiating clearance of a cholinesterase inhibitor, the method comprising administering a pharmaceutical combination of claim 148 to a subject in need thereof.

265. A method as set forth in claim 264 wherein said pharmaceutical combination comprises a pharmaceutical composition comprising:
a pyridostigmine cation; and
a facilitating anion, wherein:
the facilitating anion is less hydrophilic than a bromide anion; and
the pyridostigmine cation and the facilitating anion together are present in the pharmaceutical combination in a therapeutically effective amount.

266. A method as set forth in claim 264 wherein said pharmaceutical combination comprises a pharmaceutical kit comprising:
a source of a pyridostigmine cation; and
a source of a facilitating anion, wherein:
the facilitating anion is less hydrophilic than a bromide anion; and
the source of the pyridostigmine cation and the source of the facilitating anion together are present in the pharmaceutical combination in a therapeutically effective amount.

267. A method for potentiating clearance of a cholinesterase inhibitor, the method comprising administering a pharmaceutical combination of claim 180 to a subject in need thereof.

268. A method as set forth in claim 267 wherein said pharmaceutical combination comprises a pharmaceutical composition comprising:
a pyridostigmine cation; and
a facilitating anion, wherein:
the facilitating anion is less hydrophilic than a bromide anion;
the pyridostigmine cation and the facilitating anion are, in combination, suitable for oral ingestion;
the pyridostigmine cation and the facilitating anion are capable of forming a mixture comprising a pyridostigmine cation and a facilitating anion within the gastrointestinal tract of a subject upon ingestion by the subject; and
the pyridostigmine cation and the facilitating anion together are present in the pharmaceutical combination in a therapeutically effective amount.

269. A method as set forth in claim 267 wherein said pharmaceutical combination comprises a pharmaceutical kit comprising:
a source of a pyridostigmine cation; and
a source of a facilitating anion, wherein:
the facilitating anion is less hydrophilic than a bromide anion;
the source of the pyridostigmine cation and the source of the facilitating anion are, in combination, suitable for oral ingestion;
the source of the pyridostigmine cation and the source of the facilitating anion are capable of forming a mixture comprising a pyridostigmine cation and a facilitating anion within the gastrointestinal tract of a subject upon ingestion by the subject; and
the source of the pyridostigmine cation and the source of the facilitating anion together are present in the pharmaceutical combination in a therapeutically effective amount.

270. A method for potentiating clearance of a cholinesterase inhibitor, the method comprising administering a pharmaceutical combination of claim 212 to a subject in need thereof.

271. A method as set forth in claim 270 wherein said pharmaceutical combination comprises a pharmaceutical composition comprising:
a pyridostigmine cation and an anion selected from the group consisting of: 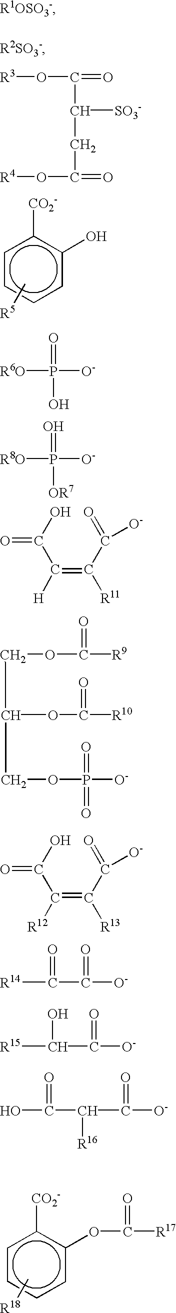
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

272. A method as set forth in claim 270 wherein said pharmaceutical combination comprises a pharmaceutical kit comprising:
a source of a pyridostigmine cation; and
a source of an anion, wherein:
the anion comprises an anion selected from the group consisting of: 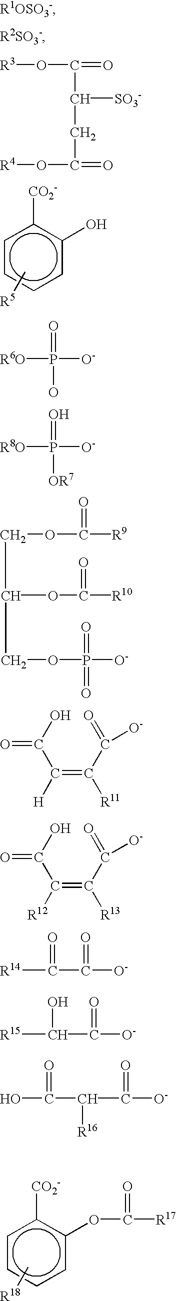
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

273. A method of potentiating clearance of a cholinesterase inhibitor, the method comprising administering a pharmaceutical combination of claim 231 to a subject in need thereof.

274. A method as set forth in claim 273 wherein said pharmaceutical combination comprises a pharmaceutical composition comprising:
a potentiating agent; and
a facilitating anion, wherein:
the facilitating anion is less hydrophilic than a chloride anion; and
the potentiating agent and the facilitating anion together are present in the pharmaceutical combination in a therapeutically effective amount.

275. A method as set forth in claim 273 wherein said pharmaceutical combination comprises a pharmaceutical kit comprising:
a source of a potentiating agent; and
a source of a facilitating anion, wherein:
the facilitating anion is less hydrophilic than a chloride anion; and
the source of the potentiating agent and the source of the facilitating anion together are present in the pharmaceutical combination in a therapeutically effective amount.

276. A pharmaceutical combination useful for treating nerve conditions such as pseudoobstruction of the bowel, paralytic ileus and/or urinary retention, or myasthenia gravis, the pharmaceutical combination comprising:
a neostigmine cation or a source of a neostigmine cation; and
a facilitating anion or a source of a facilitating anion, wherein:
the facilitating anion is less hydrophilic than a bromide anion; and
the neostigmine cation or the source of the neostigmine cation and the facilitating anion or the source of the facilitating anion together are present in the pharmaceutical combination in a therapeutically effective amount.

277. A pharmaceutical combination as set forth in claim 276 wherein the combination comprises a pharmaceutical composition comprising:
a neostigmine cation; and
a facilitating anion, wherein:
the facilitating anion is less hydrophilic than a bromide anion; and
the neostigmine cation and the facilitating anion together are present in the pharmaceutical combination in a therapeutically effective amount.

278. A pharmaceutical combination as set forth in claim 277, wherein the combination is used to treat myasthenia gravis.

279. A pharmaceutical combination as set forth in claim 277, wherein the combination is used to treat paralytic ileus and/or urinary retention or pseudoobstruction of the bowel.

280. A pharmaceutical combination as set forth in claim 277, wherein said combination further comprises a neutralizing agent.

281. A pharmaceutical combination as set forth in claim 280, wherein said neutralizing agent comprises sodium bicarbonate or sodium citrate.

282. A pharmaceutical combination as set forth in claim 280, wherein said combination further comprises a buffering agent.

283. A pharmaceutical combination as set forth in claim 282, wherein said buffering agent comprises citric acid.

284. A pharmaceutical combination as set forth in claim 277, wherein said facilitating anion has an organic/aqueous phase distribution equilibrium constant of greater than about 320 when introduced into a mixture comprising water, 1-decanol, methyltridecylammonium chloride, and a methyltridecylammonium salt.

285. A pharmaceutical combination as set forth in claim 277 wherein the molar ratio of said facilitating anion to said neostigmine cation is from about 0.5 to about 2.

286. A pharmaceutical combination as set forth in claim 285 wherein the molar ratio of said facilitating anion to said neostigmine cation is from about 1.0 to about 1.5.

287. A pharmaceutical combination as set forth in claim 285 wherein the molar ratio of said facilitating anion to said neostigmine cation is from about 1.0 to about 1.1.

288. A pharmaceutical combination as set forth in claim 277, wherein, when the combination is orally administered to a human, the neostigmine cation is absorbed into the bloodstream from the gastrointestinal tract.

289. A pharmaceutical combination as set forth in claim 277, wherein said facilitating anion comprises an anion selected from the group consisting of alkylsulfate, alkylsulfonate, alkylsulfosuccinate, salicylate, alkylsalicylate, alkylphosphate, dialkylphosphate, and dialkanoylphosphatidate.

290. A pharmaceutical composition as set forth in claim 289, wherein said facilitating anion comprises di(2-ethylhexyl)sulfosuccinate, salicylate, di(2-ethylhexyl) phosphate, hexadecylsulfonate, or dipalmitoyl phosphatidate.

291. A pharmaceutical composition as set forth in claim 290, wherein said facilitating anion comprises salicylate.

292. A pharmaceutical combination as set forth in claim 277, wherein said combination comprises a compound which comprises both said neostigmine cation and said facilitating anion.

293. A pharmaceutical combination as set forth in claim 277, wherein said combination comprises neostigmine di(2-ethylhexyl)sulfosuccinate, neostigmine salicylate, neostigmine acetylsalicylate, neostigmine lauryl sulfate, neostigmine ne di(2-ethylhexyl)phosphate, or neostigmine hexadecylsulfonate.

294. A pharmaceutical combination as set forth in claim 277, wherein said pharmaceutical combination comprises a pharmaceutical composition comprising at least 2 types of facilitating anions.

295. A pharmaceutical combination as set forth in claim 277, wherein said combination is in a form comprising a tablet or a capsule.

296. A pharmaceutical combination as set forth in claim 277, wherein said combination is in a form comprising a solution or suspension.

297. A pharmaceutical combination as set forth in claim 277, wherein said combination is in a form suitable for administering via injection.

298. A pharmaceutical combination as set forth in claim 297, wherein said combination is suitable for intravenous or intramuscular injection.

299. A pharmaceutical combination as set forth in claim 297 further comprising a buffer.

300. A pharmaceutical combination as set forth in claim 299 further comprising a bulking, dispersing, wetting or suspending agent.

301. A pharmaceutical combination as set forth in claim 276 wherein the combination comprises a pharmaceutical kit comprising:
a source of a neostigmine cation; and
a source of a facilitating anion, wherein:
the facilitating anion is less hydrophilic than a bromide anion; and
the source of the neostigmine cation and the source of the facilitating anion together are present in the pharmaceutical combination in a therapeutically effective amount.

302. A pharmaceutical combination as set forth in claim 301, wherein said combination comprises a pharmaceutical kit comprising at least 2 separate unit dosages, said unit dosages independently comprising said neostigmine cation and said facilitating anion.

303. A pharmaceutical combination as set forth in claim 301, wherein said combination further comprises a source of a neutralizing agent and/or a buffering agent.

304. A pharmaceutical combination as set forth in claim 303, wherein said combination comprises a pharmaceutical kit comprising at least three separate unit dosages, said unit dosages independently comprising the neostigmine cation, the facilitating anion, and the neutralizing agent and/or buffering agent.

305. A pharmaceutical combination as set forth in claim 303 wherein said neutralizing agent comprises sodium bicarbonate or sodium citrate and said buffering agent comprises citric acid.

306. A pharmaceutical combination as set forth in claim 305 wherein said sources of said neutralizing agent and said buffering agent are in combination and comprise a commercially available Alka Seltzer® tablet.

307. A pharmaceutical combination as set forth in claim 301, wherein said pharmaceutical combination comprises a pharmaceutical kit comprising at least 2 types of facilitating anions.

308. A pharmaceutical combination as set forth in claim 276, wherein the combination comprises:
a neostigmine cation or a source of a neostigmine cation; and
a facilitating anion or a source of a facilitating anion, wherein:
the facilitating anion is less hydrophilic than a bromide anion;
the neostigmine cation or the source of the neostigmine cation and the facilitating anion or the source of the facilitating anion are, in combination, suitable for oral ingestion;
the neostigmine cation or the source of the neostigmine cation and the facilitating anion or the source of the facilitating anion are capable of forming a mixture comprising a neostigmine cation and a facilitating anion within the gastrointestinal tract of a subject upon ingestion by the subject; and
the neostigmine cation or the source of the neostigmine cation and the facilitating anion or the source of the facilitating anion together are present in the pharmaceutical combination in a therapeutically effective amount.

309. A pharmaceutical combination as set forth in claim 308 wherein the combination comprises a pharmaceutical composition comprising:
a neostigmine cation; and
a facilitating anion, wherein:
the facilitating anion is less hydrophilic than a bromide anion;
the neostigmine cation and the facilitating anion are, in combination, suitable for oral ingestion;
the neostigmine cation and the facilitating anion are capable of forming a mixture comprising a neostigmine cation and a facilitating anion within the gastrointestinal tract of a subject upon ingestion by the subject; and
the neostigmine cation and the facilitating anion together are present in the pharmaceutical combination in a therapeutically effective amount.

310. A pharmaceutical combination as set forth in claim 309, wherein the combination is used to treat myasthenia gravis.

311. A pharmaceutical combination as set forth in claim 309, wherein the combination is used to treat paralytic ileus and/or urinary retention or pseudoobstruction of the bowel.

312. A pharmaceutical combination as set forth in claim 309, wherein said combination further comprises a neutralizing agent.

313. A pharmaceutical combination as set forth in claim 312, wherein said neutralizing agent comprises sodium bicarbonate or sodium citrate.

314. A pharmaceutical combination as set forth in claim 312, wherein said combination further comprises a buffering agent.

315. A pharmaceutical combination as set forth in claim 314, wherein said buffering agent comprises citric acid.

316. A pharmaceutical combination as set forth in claim 309, wherein said facilitating anion has an organic/aqueous phase distribution equilibrium constant of greater than about 320 when introduced into a mixture comprising water, 1-decanol, methyltridecylammonium chloride, and a methyltridecylammonium salt.

317. A pharmaceutical combination as set forth in claim 309 wherein the molar ratio of said facilitating anion to said neostigmine cation is from about 0.5 to about 2.

318. A pharmaceutical combination as set forth in claim 317 wherein the molar ratio of said facilitating anion to said neostigmine cation is from about 1.0 to about 1.5.

319. A pharmaceutical combination as set forth in claim 1317 wherein the molar ratio of said facilitating anion to said neostigmine cation is from about 1.0 to about 1.1.

320. A pharmaceutical combination as set forth in claim 309, wherein, when the combination is orally administered to a human, the neostigmine cation is absorbed into the bloodstream from the gastrointestinal tract.

321. A pharmaceutical combination as set forth in claim 309, wherein said facilitating anion comprises an anion selected from the group consisting of alkylsulfate, alkylsulfonate, alkylsulfosuccinate, salicylate, alkylsalicylate, alkylphosphate, dialkylphosphate, and dialkanoylphosphatidate.

322. A pharmaceutical composition as set forth in claim 321, wherein said facilitating anion comprises di(2-ethylhexyl)sulfosuccinate, salicylate, di(2-ethylhexyl) phosphate, hexadecylsulfonate, or dipalmitoyl phosphatidate.

323. A pharmaceutical composition as set forth in claim 322, wherein said facilitating anion comprises salicylate.

324. A pharmaceutical combination as set forth in claim 309, wherein said combination comprises a compound which comprises both said neostigmine cation and said facilitating anion.

325. A pharmaceutical combination as set forth in claim 309, wherein said combination comprises neostigmine di(2-ethylhexyl)sulfosuccinate, neostigmine salicylate, neostigmine acetylsalicylate, neostigmine lauryl sulfate, neostigmine ne di(2-ethylhexyl)phosphate, or neostigmine hexadecylsulfonate.

326. A pharmaceutical combination as set forth in claim 309, wherein said pharmaceutical combination comprises a pharmaceutical composition comprising at least 2 types of facilitating anions.

327. A pharmaceutical combination as set forth in claim 309, wherein said combination is in a form comprising a tablet or a capsule.

328. A pharmaceutical combination as set forth in claim 309, wherein said combination is in a form comprising a solution or suspension.

329. A pharmaceutical combination as set forth in claim 309, wherein said combination is in a form suitable for administering via injection.

330. A pharmaceutical combination as set forth in claim 329, wherein said combination is suitable for intravenous or intramuscular injection.

331. A pharmaceutical combination as set forth in claim 329 further comprising a buffer.

332. A pharmaceutical combination as set forth in claim 331 further comprising a bulking, dispersing, wetting or suspending agent.

333. A pharmaceutical combination as set forth in claim 308 wherein the combination comprises a pharmaceutical kit comprising:
a source of a neostigmine cation; and
a source of a facilitating anion, wherein:
the facilitating anion is less hydrophilic than a bromide anion;
the source of the neostigmine cation and the source of the facilitating anion are, in combination, suitable for oral ingestion;
the source of the neostigmine cation and the source of the facilitating anion are capable of forming a mixture comprising a neostigmine cation and a facilitating anion within the gastrointestinal tract of a subject upon ingestion by the subject; and
the source of the neostigmine cation and the source of the facilitating anion together are present in the pharmaceutical combination in a therapeutically effective amount.

334. A pharmaceutical combination as set forth in claim 333, wherein said combination comprises a pharmaceutical kit comprising at least 2 separate unit dosages, said unit dosages independently comprising said neostigmine cation and said facilitating anion.

335. A pharmaceutical combination as set forth in claim 333, wherein said combination further comprises a source of a neutralizing agent and/or a buffering agent.

336. A pharmaceutical combination as set forth in claim 335, wherein said combination comprises a pharmaceutical kit comprising at least three separate unit dosages, said unit dosages independently comprising the neostigmine cation, the facilitating anion, and the neutralizing agent and/or buffering agent.

337. A pharmaceutical combination as set forth in claim 335 wherein said neutralizing agent comprises sodium bicarbonate or sodium citrate and said buffering agent comprises citric acid.

338. A pharmaceutical combination as set forth in claim 337 wherein said sources of said neutralizing agent and said buffering agent are in combination and comprise a commercially available Alka Seltzer® tablet.

339. A pharmaceutical combination as set forth in claim 333, wherein said pharmaceutical combination comprises a pharmaceutical kit comprising at least 2 types of facilitating anions.

340. A pharmaceutical combination useful for treating nerve conditions such as pseudoobstruction of the bowel, paralytic ileus and/or urinary retention, or myasthenia gravis, the pharmaceutical combination comprising:
a neostigmine cation or a source of a neostigmine cation; and
an anion or a source of an anion, wherein:
the anion comprises an anion selected from the group consisting of: 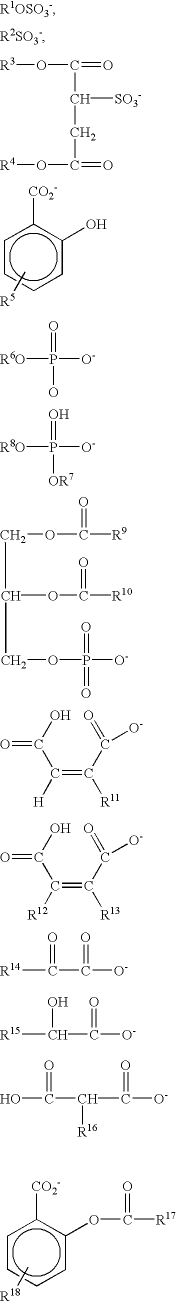
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

341. A pharmaceutical combination as set forth in claim 340 wherein the combination comprises a pharmaceutical composition comprising:
a neostigmine cation; and
an anion, wherein:
the anion comprises an anion selected from the group consisting of: 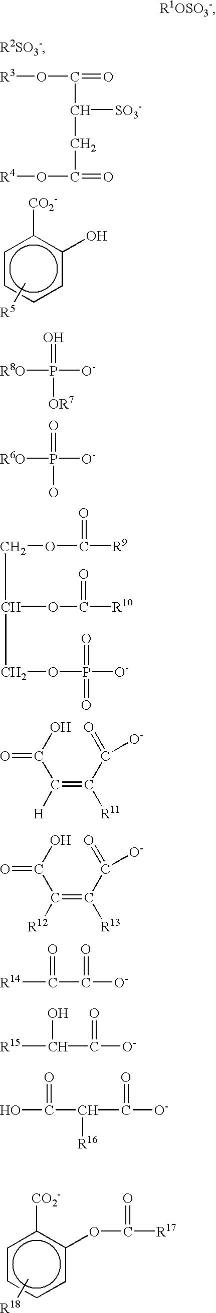
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

342. A pharmaceutical combination as set forth in claim 341, wherein the anion comprises R¹OSO₃⁻ and R¹ is hydrocarbyl or substituted hydrocarbyl.

343. A pharmaceutical combination as set forth in claim 342, wherein R¹ is hydrocarbyl.

344. A pharmaceutical combination as set forth in claim 341, wherein the facilitating anion comprises the formula: 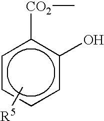
wherein R⁵ is hydrocarbyl or substituted hydrocarbyl.

345. A pharmaceutical combination as set forth in claim 344, wherein R⁵ is hydrocarbyl.

346. A pharmaceutical combination as set forth in claim 341, wherein the anion comprises a compound of the the formula: 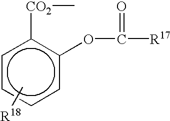
wherein:
R¹⁷ is hydrocarbyl or substituted hydrocarbyl; and
R¹⁸ is hydrogen, hydrocarbyl, or substituted hydrocarbyl.

347. A pharmaceutical combination as set forth in claim 346, wherein R¹⁷ is hydrocarbyl and R¹⁸ is hydrogen.

348. A pharmaceutical combination as set forth in claim 346, wherein the pharmaceutical composition comprises aspirin.

349. A pharmaceutical combination as set forth in claim 346, wherein the pharmaceutical composition is in a form suitable for administering orally.

350. A pharmaceutical combination as set forth in claim 340 wherein the combination comprises a pharmaceutical kit comprising:
a source of a neostigmine cation; and
a source of an anion, wherein:
the anion comprises an anion selected from the group consisting of: 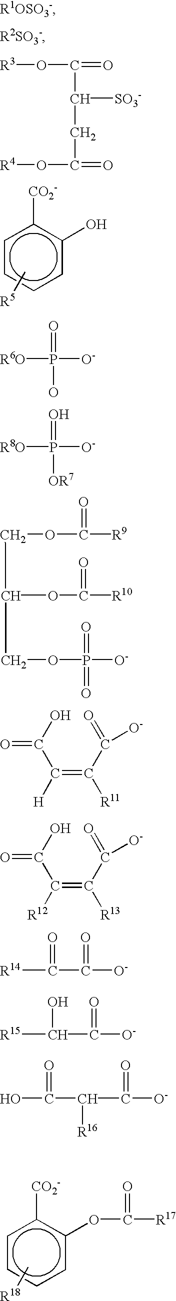
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

351. A pharmaceutical combination as set forth in claim 350, wherein the anion comprises R¹OSO₃⁻ and R¹ is hydrocarbyl or substituted hydrocarbyl.

352. A pharmaceutical combination as set forth in claim 351, wherein R¹ is hydrocarbyl.

353. A pharmaceutical combination as set forth in claim 352, wherein the anion comprises a compound of the formula: 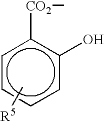
wherein R⁵ is hydrocarbyl or substituted hydrocarbyl.

354. A pharmaceutical combination as set forth in claim 353, wherein R⁵ is hydrocarbyl.

355. A pharmaceutical combination as set forth in claim 350, wherein the anion comprises a compound having the formula: 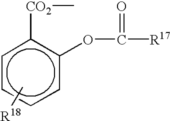
wherein:
R¹⁷ is hydrocarbyl or substituted hydrocarbyl; and
R¹⁸ is hydrogen, hydrocarbyl, or substituted hydrocarbyl.

356. A pharmaceutical combination as set forth in claim 355, wherein R¹⁷ is hydrocarbyl and R¹⁸ is hydrogen.

357. A pharmaceutical combination as set forth in claim 355, wherein the pharmaceutical composition comprises aspirin.

358. A pharmaceutical combination as set forth in claim 355, wherein the pharmaceutical composition is in a form suitable for administering orally.

359. A method for treating nerve conditions such as pseudoobstruction of the bowel, paralytic ileus and/or urinary retention, or myasthenia gravis, the method comprising administering a pharmaceutical combination of claim 276 to a subject in need thereof.

360. A method as set forth in claim 359 wherein said pharmaceutical combination comprises a pharmaceutical composition comprising:
a neostigmine cation; and
a facilitating anion, wherein:
the facilitating anion is less hydrophilic than a chloride anion; and
the neostigmine cation and the facilitating anion together are present in the pharmaceutical combination in a therapeutically effective amount.

361. A method as set forth in claim 359 wherein said pharmaceutical combination comprises a pharmaceutical kit comprising:
a source of a neostigmine cation; and
a source of a facilitating anion, wherein:
the facilitating anion is less hydrophilic than a chloride anion; and
the source of the neostigmine cation and the source of the facilitating anion together are present in the pharmaceutical combination in a therapeutically effective amount.

362. A method for treating nerve conditions such as pseudoobstruction of the bowel, paralytic ileus and/or urinary retention, or myasthenia gravis, the method comprising administering a pharmaceutical combination of claim 308 to a subject in need thereof.

363. A method as set forth in claim 362 wherein said pharmaceutical combination comprises a pharmaceutical composition comprising:
a neostigmine cation; and
a facilitating anion, wherein:
the facilitating anion is less hydrophilic than a chloride anion;
the neostigmine cation and the facilitating anion are, in combination, suitable for oral ingestion;
the neostigmine cation and the facilitating anion are capable of forming a mixture comprising a neostigmine cation and a facilitating anion within the gastrointestinal tract of a subject upon ingestion by the subject; and
the neostigmine cation and the facilitating anion together are present in the pharmaceutical combination in a therapeutically effective amount.

364. A method as set forth in claim 362 wherein said pharmaceutical combination comprises a pharmaceutical kit comprising:
a source of a neostigmine cation; and
a source of a facilitating anion, wherein:
the facilitating anion is less hydrophilic than a chloride anion;
the source of the neostigmine cation and the source of the facilitating anion are, in combination, suitable for oral ingestion;
the source of the neostigmine cation and the source of the facilitating anion are capable of forming a mixture comprising a neostigmine cation and a facilitating anion within the gastrointestinal tract of a subject upon ingestion by the subject; and
the source of the neostigmine cation and the source of the facilitating anion together are present in the pharmaceutical combination in a therapeutically effective amount.

365. A method for treating nerve conditions such as pseudoobstruction of the bowel, paralytic ileus and/or urinary retention, or myasthenia gravis, the method comprising administering a pharmaceutical combination of claim 340 to a subject in need thereof.

366. A method as set forth in claim 365 wherein said pharmaceutical combination comprises a pharmaceutical composition comprising:
a neostigmine cation; and
an anion, wherein:
the anion comprises an anion selected from the group consisting of: 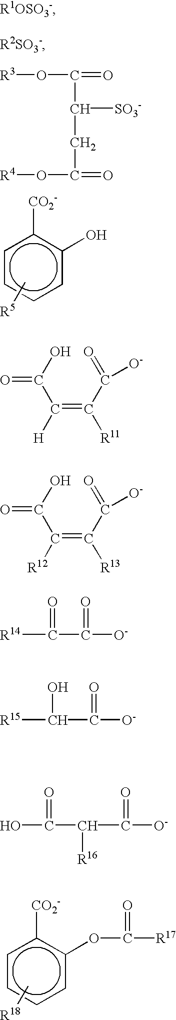
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

367. A method as set forth in claim 365 wherein said pharmaceutical combination comprises a pharmaceutical kit comprising:
a source of a neostigmine cation; and
a source of an anion, wherein:
the anion comprises an anion selected from the group consisting of: 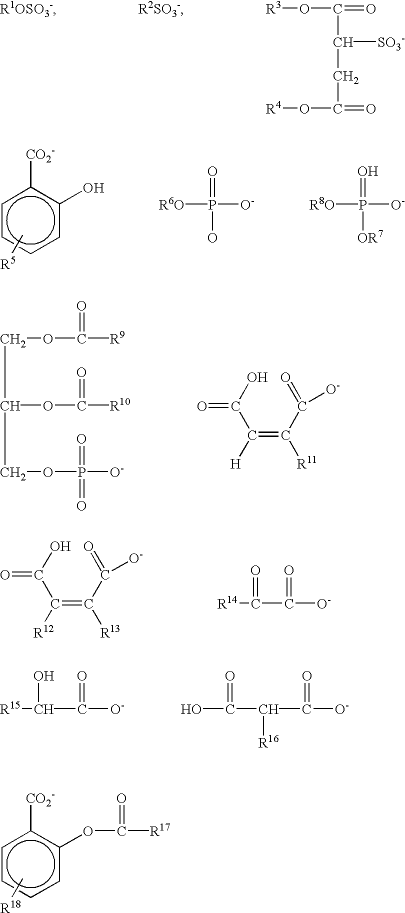
a pseudo-icosahedral carboranes anion (CB₁₁H₁₂⁻), and
a substituted pseudo-icosahedral carborane anion, wherein R¹, R², R³, R⁴, R⁶, R⁷, R⁸, R⁹, R¹⁰, R¹¹, R¹², R¹³, R¹⁴, R¹⁵, R¹⁶, R¹⁷, and the substituent (or substituents) of the substituted pseudo-icosahedral carborane anion are independently hydrocarbyl or substituted hydrocarbyl; and R⁵ and R¹⁸ are independently hydrogen, hydrocarbyl, or substituted hydrocarbyl.

368. A process for the preparation of a salt of a quaternary ammonium cation and an anion that is more hydrophobic than chloride ion, the process comprising:
mixing an aqueous solution of a mineral acid salt of the cation with a source of alkali metal or alkaline earth metal salt of the anion; and
contacting the resulting mixture with a substantially water-immiscible organic solvent, thereby transferring the salt of the quaternary ammonium cation and the more hydrophobic anion to the solvent phase and producing an organic extract comprising the transferred salt.

369. A process as set forth in claim 368 wherein the transferred salt is recovered from the extract.

370. A process as set forth in claim 369 wherein recovery of transferred salt from the extract comprises:
dehydrating the extract; and
removing solvent from the dehydrated extract to yield a residue comprising the salt of the quaternary ammonium cation and the more hydrophobic anion.

371. A process as set forth in claim 368 wherein the quaternary ammonium cation is selected from the group consisting of 2-PAM, pyridostigmine and neostigmine.

372. A process as set forth in claim 368 wherein the more hydrophobic anion is selected from the group consisting of alkylsulfonate, alkylsulfosuccinate, alkylphosphate, dialkylphosphate, dialkanoylphosphatidate, dialkylsulfosuccinate, salicylate, and alkylsulfate.

373. A process as set forth in claim 368 wherein the mineral acid salt is selected from the group consisting of halide and sulfate salts.

374. A process as set forth in claim 373 wherein the mineral acid salt comprises a chloride salt.

375. The process as set forth in claim 368 wherein the anion is more hydrophobic than toluenesulfonate.

376. A pharmaceutical composition comprising 2-PAM di(2-ethylhexyl)sulfosuccinate

377. A pharmaceutical composition comprising 2-PAM salicylate

378. A pharmaceutical composition comprising 2-PAM di(2-ethylhexyl)phosphate

379. A pharmaceutical composition comprising 2-PAM @ lauryl sulfate

380. A pharmaceutical composition comprising 2-PAM hexadecylsulfonate

381. A pharmaceutical composition comprising 2-PAM acetyl salicylate

382. A pharmaceutical composition comprising pyridostigmine hexadecylsulfonate

383. A pharmaceutical composition comprising pyridostigmine di(2-ethylhexyl)sulfosuccinate

384. A pharmaceutical composition comprising pyridostigmine salicylate

385. A pharmaceutical composition comprising pyridostigmine di(2-ethylhexyl)phosphate

386. A pharmaceutical composition comprising pyridostigmine lauryl sulfate

387. A pharmaceutical composition comprising pyridostigmine acetyl salicylate

388. A pharmaceutical composition comprising neostigmine hexadecylsulfonate

389. A pharmaceutical composition comprising neostigmine di(2-ethylhexyl)sulfosuccinate

390. A pharmaceutical composition comprising neostigmine salicylate

391. A pharmaceutical composition comprising neostigmine di(2-ethylhexyl)phosphate

392. A pharmaceutical composition comprising neostigmine lauryl sulfate

393. A pharmaceutical composition comprising neostigmine acetyl salicylate
